(19) United States
(12) Patent Application Publication
(10) Pub. No.: US 2021/0393674 A1
(43) Pub. Date: Dec. 23, 2021

(54) OZONOLYSIS FOR ACTIVATION OF COMPOUNDS AND DEGRADATION OF OZONE

(71) Applicant: AIR CROSS, INC., San Diego (CA)

(72) Inventor: Christopher Duke, San Diego, CA (US)

(21) Appl. No.: 15/761,101

(22) PCT Filed: Sep. 19, 2016
(86) PCT No.: PCT/US16/52529

§ 371 Date: Sep. 20, 2019

Related U.S. Application Data
(60) Provisional application No. 62/237,699, filed on Oct. 6, 2015.
(60) Provisional application No. 62/221,030, filed on Sep. 20, 2015.

(51) Int. Cl. A61K 33/00; A61K 31/136; A61K 31/704; A61K 31/66; A61K 31/121; A01N 33/12; A01P 1/00; C07C 51/34; C07C 45/40; C07C 225/36; C07H 15/244

(52) U.S. Cl. CPC A61K 33/00; A61K 31/136; A61K 31/704; A61K 31/66; A61K 31/121; C07C 2603/24; A01P 1/00; C07C 51/34; C07C 45/40; C07C 225/36; C07H 15/244; A01N 33/12

(57) ABSTRACT

Provided is an inactive compound that is activated by reaction with ozone into an active compound having a carbonyl oxygen. Also provided is a method of activating the above inactive compounds. Further provided is a method of treating a disease or condition in a subject using the above compound at a site that is not exposed to atmospheric ozone. Additionally provided is a method of determining internal ozonolysis in a subject using the above compound. Also provided is a molecule less than 1000 mw, having a double bond that is reactive with ozone, and forms a nontoxic compound after reacting with ozone. Further provided is a method of degrading ozone.

### CROSS-REFERENCE TO RELATED APPLICATIONS

This application claims the benefit of U.S. Provisional Application No. 62/221,030, filed Sep. 20, 2015 and U.S. Provisional Application No. 62/237,699, filed Oct. 6, 2015, both incorporated by reference herein in their entirety.

### BACKGROUND OF THE INVENTION

### (1) Field of the Invention

The present application generally relates to chemical reactions with ozone. More specifically, the application is directed to compounds and methods of using ozonolysis reactions to activate an inactive compound or to degrade ozone.

### (2) Description of the Related Art

As discussed in U.S. Provisional Application 62/034,864 and PCT Publication WO 2016/023015 (both incorporated by reference), ozone is a triatomic molecule composed of three oxygen atoms. It is formed from diatomic oxygen (O₂) by the action of sunlight, ultraviolet light or an electrical discharge. Scheme 1 illustrates the resonance structures of triatomic ozone (03).

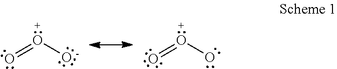

Ozone is formed in the atmosphere by the action of sunlight, ultraviolet light or an electrical discharge such as lightning on oxygen in the air. It is also formed when an electrical apparatus produces sparks in the air.

Ozone reacts with alkenes and alkynes to form organic compounds in a process known as ozonolysis. The multiple bonds in these compounds are oxidized by the action of ozone to provide compounds in which the double bonds form a carbonyl group. The outcome of the reaction depends on the type of multiple bonds being oxidized. For example, alkenes can be oxidized by ozone to form aldehydes, ketones, carboxylic acids, esters, amides, enones, acyl halides, imides, acid anhydrides, 1,3-dicarbonyls, carbamates, carbazides, carbazones, carboxylates, cyclic imides, formates, furazones, hydrazines, hydroxamates, isocyanates, lactams, lactones, semicarbazones, ureas, thiocarbamates, dithiocarbamates, etc. Often, two aldehydes and/or ketones are produced when the olefinic compound is appropriately substituted. Scheme 2 illustrates an ozonolysis reaction between a carbon-carbon double bond and ozone. The reaction provides two carbonyl containing compounds depending upon the R substituents.

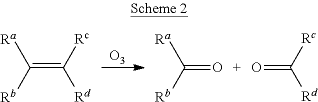

Ozone in the air may be toxic to human beings and animals. According to Occupational Safety and Health Administration (OSHA), the permissible maximal average concentration of ozone in the air should be no more than 0.1 ppm when breathing air. Many apparatuses for industrial use are manufactured in accordance with these standards. Ozone has a characteristic odor, which is noticeable even at concentrations as low as 0.01 to 0.02 ppm. When the concentration of ozone increases to about 0.05 ppm, it has an unpleasant odor; and when the concentration exceeds 0.1 ppm, it is irritating to the mucous membranes of the eyes and respiratory organs. Ozone is also a powerful oxidizing agent which oxidizes and deteriorates organic materials. Therefore, it is desirable that the concentration of ozone be kept as low as possible.

Ozone is used in industry for the sterilization, deodorization and decolorization of water and for the treatment of raw sewage. These applications often require the use of ozone in concentrations as high as 500-2500 ppm. For example, to sterilize water, 1 to 3 g of ozone is bubbled into 1 cubic meter of water. Most of the ozone blown into water is decomposed, however, some of the residual ozone can be discharged from the water into the air. Since the concentration of the discharged ozone in the air may be as high as 1 ppm, it is necessary to decompose the discharged ozone before it spreads into the air for the safety to human beings and for the protection of the environment.

Since ozone is toxic to human beings when its concentration in the air is high, various methods have been proposed to decrease its concentration. For example, filters made of activated carbon and filters containing various catalysts, such as metal oxides of manganese, copper, silver and cobalt, have been employed for decomposing ozone. If the density of the materials in these filters is high, the absorption of ozone and its decomposition efficiency is increased. However, the higher density of these materials slows the flow rate of the air through the filter. By contrast, if the density of the materials in the filter is decreased, the absorption of ozone and the ozone decomposition efficiency are decreased.

Various polymers and terpenoid compounds have also been used to control ozone levels. For example, a rubber olefin polymer containing double bond groups has been used for decomposing ozone generated from an electrophotographic copying machine. Terpenoid compounds capable of decomposing ozone, such as linalool, linalool ester, citral and the like, in various solutions and gels have also been used. In addition, paints containing a variety of organic materials have been proposed. However, the decomposition efficiency is not high enough for use in practice. Furthermore, the by-products formed after decomposition of the ozone has not been fully characterized in these cases. Therefore, it is unclear whether exposure to these by-products affect a person's health, and whether there are any negative environmental impacts.

Therefore, there remains a need in the art for new compounds, compositions and methods for removing and/or controlling ozone levels without having a negative impact on humans, animals and the environment, wherein the by-products formed after decomposition of the ozone is safe and fully characterized. The present invention addresses that need by providing small molecule compounds that degrade ozone, leaving known, nontoxic by-products.

There are various methods for activing inactive compounds. A well-known example is prodrugs, which are pharmaceuticals that are inactive when administered and become activated by body metabolism. Another example is caged compounds that are activated by light (see, e.g., Ellis-Davies, 2007, Nat. Methods 4:619-628). There is a need for additional means for activating inactive compounds. The present invention addresses those needs by providing inactive compounds that are activated by exposure to ozone.

### BRIEF SUMMARY OF THE INVENTION

The present invention provides compounds and methods for degrading ozone and for using ozone to activate inactive compounds. Thus, in some embodiments, the present invention is directed to an inactive compound that is activated by reaction with ozone into an active compound having a carbonyl oxygen.

Also provided is a method of activating the above inactive compounds. The method comprises exposing the inactive compound with ozone for a time sufficient to activate the compound.

Additionally provided is a method of treating a disease or condition in a subject. The method comprises administering an inactive pharmaceutical compound that is activated by reaction with ozone into an active compound having a carbonyl oxygen to the subject at a site that is not exposed to atmospheric ozone.

Further provided is a method of determining internal ozonolysis in a subject. The method comprises administering the above-described inactive compound to the subject, waiting for a time sufficient for the internal ozonolysis to take place, then assaying for the active compound X═O.

Also provided is a molecule less than 9000 mw, having a double bond that is reactive with ozone, and forms a nontoxic compound after reacting with ozone.

Additionally provided is a method of degrading ozone. The method comprises exposing the above molecule to ozone for a time sufficient to degrade the ozone.

### DETAILED DESCRIPTION OF THE INVENTION

As used herein, the singular forms “a”, “an” and “the” are intended to include the plural forms as well, unless the context clearly indicates otherwise. Additionally, the use of “or” is intended to include “and/or”, unless the context clearly indicates otherwise.

The meanings of various terms, including chemical moieties, are as they are defined in WO 2016/023015.

The present invention provides in part inactive compounds that are activated by ozone. Since ozone is present in the air, such inactive compounds are slowly activated upon exposure to air, providing a slow-release of an active compound. Active compounds that can be usefully created from the inactive compounds includes pharmaceuticals (where the inactive compound is a prodrug), antimicrobials, fertilizers, pesticides, cosmetics, etc. as further discussed below.

In some embodiments, the present invention is directed to an inactive compound that is activated by reaction with ozone into an active compound having a carbonyl oxygen. The carbonyl oxygen in the active compound can be part of any moiety that can be formed after reaction with ozone. In various embodiments, the carbonyl oxygen in the active compound is part of an aldehyde, a ketone, a carboxylic acid, an ester, an amide, an enone, an acyl halide, an imide, an acid anhydride, a 1,3-dicarbonyl, a carbamate, a carbazide, a carbazone, a carboxylate, a cyclic imide, a formate, a furazone, a hydrazine, a hydroxamate, an isocyanate, a lactam, a lactone, a semicarbazone, a urea, a thiocarbamate, or a dithiocarbamate.

In other embodiments, the inactive compound comprises a double or triple bond that does not necessarily form a carbonyl oxygen after reaction with ozone. Nonlimiting examples of such moieties are C═N, N═N, N═S, C═S, S═S, C═P and moieties having a triple bond between any of C, N, S and P.

The inactive compound and/or the active compound are not limited to having any particular physical properties. For example they can be volatile or non-volatile in air, or fully water soluble, sparingly water soluble or non-water soluble.

In some embodiments, the inactive compound has the structure of compound I

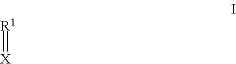

where, upon reaction with ozone, —R¹ is substituted with oxygen to form the carbonyl oxygen, forming the active compound X═O. In these embodiments, R¹ is a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl. These compounds can have 1 X or more than one X that are the same or different.

The ozonolysis reaction results in a carbonyl moiety (X═O) regardless of the side atoms or functional group in proximity to the carbonyl carbon. Thus, the reaction can result in a ketone, aldehyde, carboxylic acid, amide, etc

Many of the compounds of the present invention, besides reacting with ozone, can react with other reactive species such as singlet oxygen, dioxygen, triplet oxygen, hydroxyl radical, hydrogen peroxide, superoxides, ozone, peroxides, oxygen radicals, free radical gases, nitrogen oxides, ozonide, dioxygenyl cation, atomic oxygen, sulfur oxides, volatile organic compounds, ammonia, fine particles including those with free radicals, carbon monoxide.

In some embodiments, X is a planar compound comprising at least three aromatic rings. Nonlimiting examples include anthraquinones and anthracyclines. Another example is ethidium bromide, a nucleic acid intercalator used to treat trypanosomiasis in cattle, and having the structure

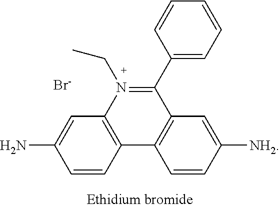

Two nonlimiting examples of a slow release ethidium bromide are compounds XXVIII and and XXIX.

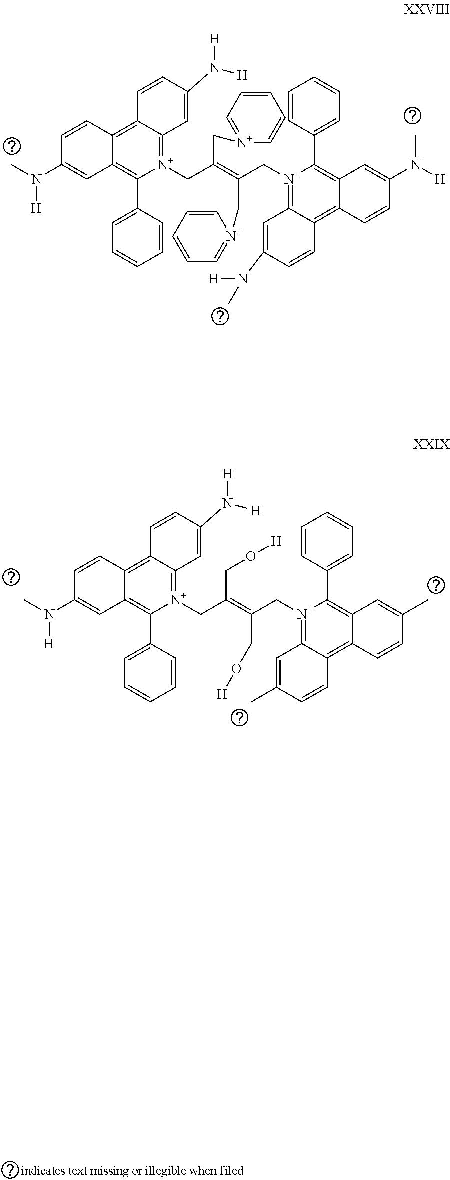

Rather than one double bond linkage between two “X” moieties, as above, any other linkage described below or in WO 2016/023015, including monomeric, oligomeric, or polymeric linkages, can be used to create the ozone-labile linkages for ethidium bromide or any of the other active compounds described herein.

With a nucleic acid intercalator like ethidium bromide and the anthraquinone and anthracycline anti-cancer pharmaceuticals described below, an effective inactivating linkage must disrupt the planar characteristic of the intercalator. The determination of whether any linkage disrupts that planar characteristic can be made without undue experimentation, by chemical modeling and testing the compound's ability to intercalate.

In some of these embodiments, X is an anthraquinone.

Among useful anthraquinones are dyes. Many anthraquinone dyes are subjected to degradation by ozone. See, e.g., Lebensaft PhD Dissertation, University of North Carolina at Greensboro, 1970. This problem can be rectified by having monomers, oligomers or polymers of the dyes using the linkages described herein, where those linkages will react with ozone and release more dye to compensate for other dye molecules that are destroyed by ozone, as well as protect the dye portion of the molecules by reacting with the ozone rather than the dye portion reacting.

Thus, in various embodiments, the anthraquinone is a dye. For example, the dye has the

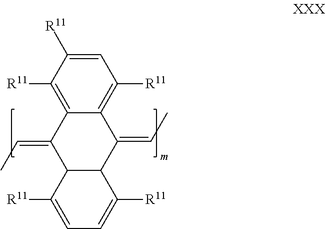

where each R¹¹ is a moiety found in a dye, and m is an integer from 2 to 100,000,000. In some embodiments, the active dye or dyes (if the structure has a mixture of two or more dyes) is at least one of the following:

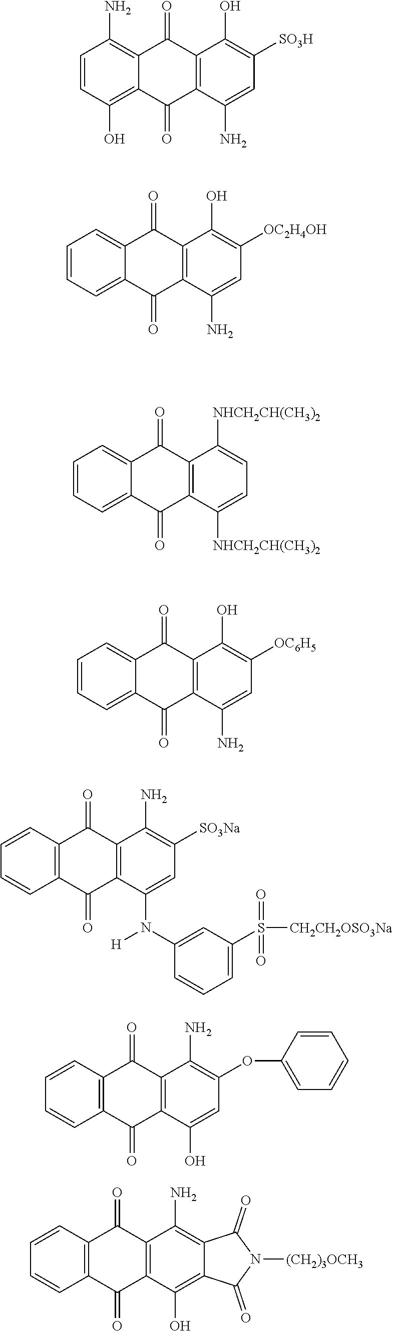

In other embodiments, the anthraquinone is a pharmaceutical. A nonlimiting example of an anthraquinone pharmaceutical is mitoxantrone, having the structure

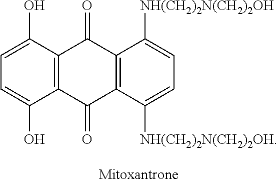

The pharmaceutical can also be a derivative of mitoxantrone, in order to provide a carbonyl oxygen to which an R¹ group can be easily joined. Nonlimiting examples of such a derivative compound is a methyl ketone or an aldehyde of mitoxantrone having the structures XXXI and XXXII.

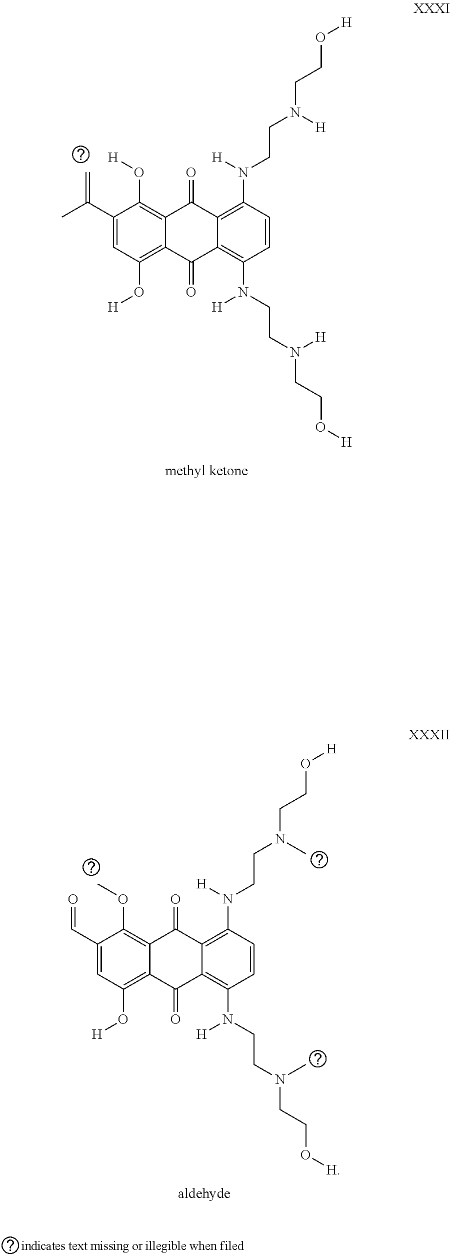

A nonlimiting example of a mitoxantrone prodrug in accordance with the present invention is the compound having the structure XXXIII.

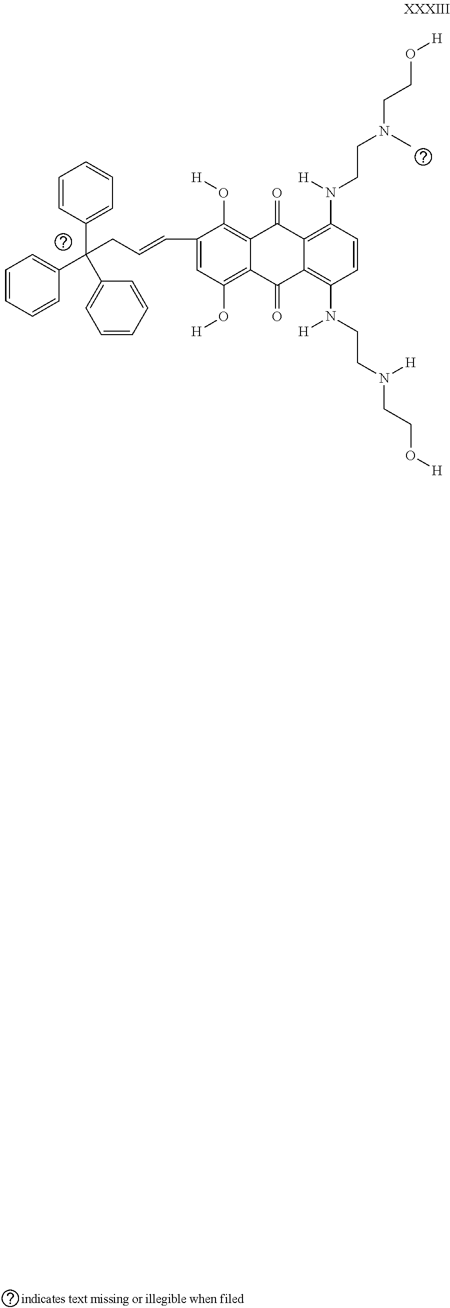 [SMILES: CN(CCO)CCNc1ccc(NCCNCCO)c2c1C(=O)c1c(O)c(/C=C/CC(c3ccccc3)(c3ccccc3)c3ccccc3)cc(O)c1C2=O]

In various embodiments, the active compound (X) is an anthracycline. Nonlimiting examples of anthracyclines are daunorubicin, doxorubicin, epirubicin, and idarubicin, as follows:

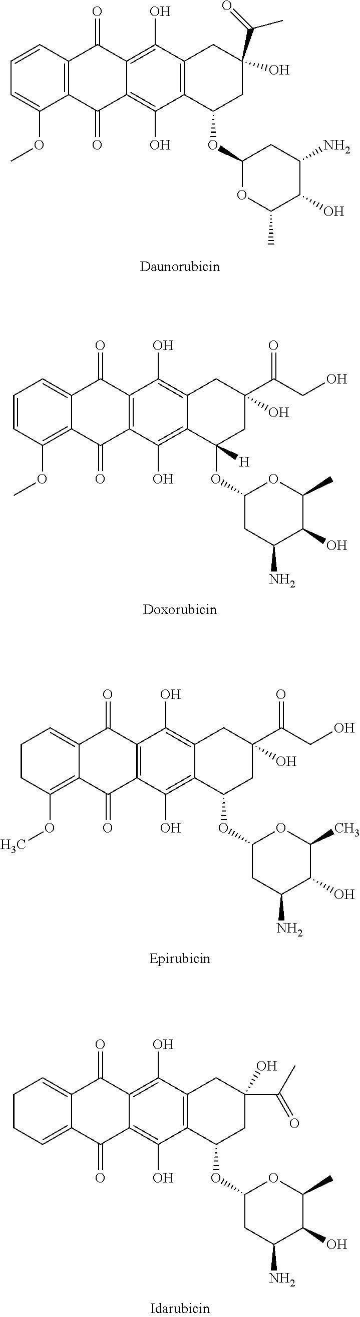 [SMILES: COc1cccc2c1C(=O)c1c(O)c3c(c(O)c1C2=O)C[C@@](O)(C(C)=O)C[C@@H]3O[C@H]1C[C@H](N)[C@H](O)[C@H](C)O1 ; COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1 ; COC1=C2C(=O)c3c(O)c4c(c(O)c3C(=O)C2=CCC1)C[C@@](O)(C(=O)CO)C[C@@H]4O[C@H]1C[C@H](N)[C@@H](O)[C@H](C)O1 ; CC(=O)[C@]1(O)Cc2c(O)c3c(c(O)c2[C@@H](O[C@H]2C[C@H](N)[C@H](O)[C@H](C)O2)C1)C(=O)C1=CCCC=C1C3=O]

Using the atomic numbering system as shown in the following aglycone structure,

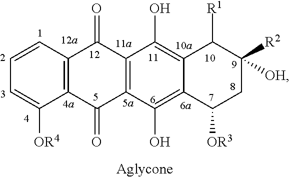

the carbonyl oxygen at the R² group at position 9 of any of the above anthracyclines, or any other anthracycline having such a carbonyl oxygen, can be easily converted to a prodrug by conjugating any inactivating moiety to that carbonyl oxygen, where ozone would convert the prodrug to the active compound.

In some embodiments, the active compound is idarubicin, with the prodrug having structure XXXIV

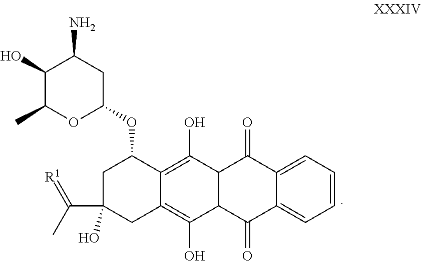

A nonlimiting example of such a compound is compound XXXV

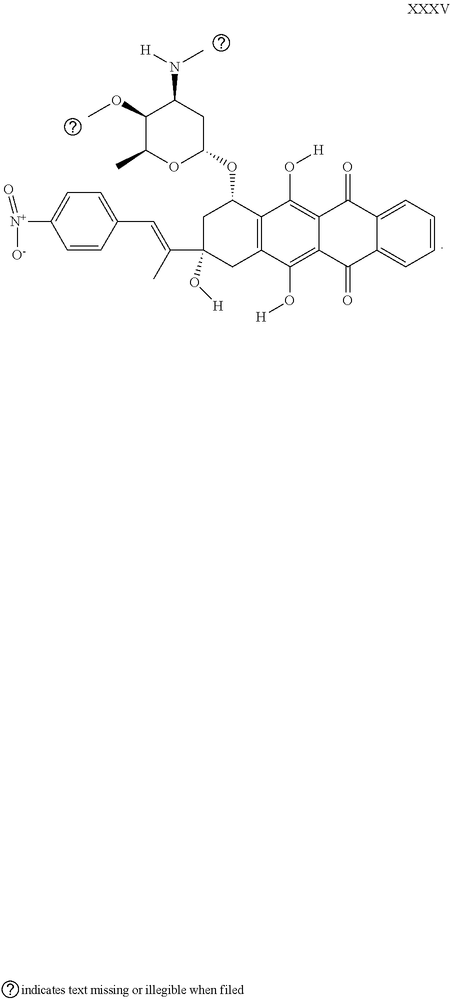 [SMILES: CN[C@H]1C[C@H](O[C@H]2C[C@](O)(/C(C)=C/c3ccc([N+](=O)[O-])cc3)Cc3c(O)c4c(c(O)c32)C(=O)c2ccccc2C4=O)O[C@@H](C)[C@H]1OC]

In some embodiments of these compounds, R¹ comprises a specific binding agent. Here, the specific binding agent can be, e.g., a peptide such as an antibody or fraction thereof comprising an antibody binding site, e.g., an Fab or an engineered or other natural protein with affinity to a cancer target (Toporkiewicz et al., 2015, Int. J. Nanomed. 10:1399-1414), or a nucleic acid such as an aptamer (Parashar, A., 2016, J. Clin. Diag. Res., 10:BE01-BE06). Any other compound described herein can comprise such a specific binding agent if appropriate, in the R¹ moiety.

The R¹ inactivating group can also comprise a nanoparticle, e.g., as described in WO 2009/038776, or a liposome.

In some embodiments of compound I above, R¹ is NR² or CR², where R² is H, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl.

In certain embodiments, the compound has the structure

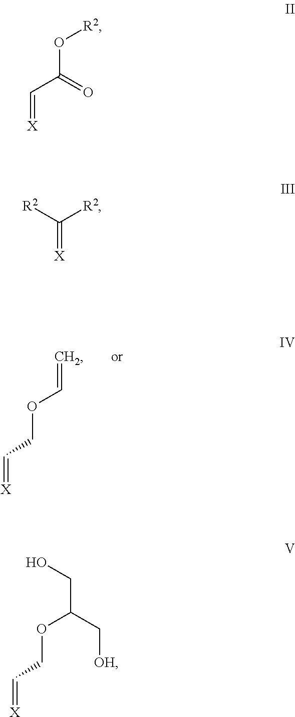

where each R² is independently hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl.

In some of those embodiments, the compound has structure of compound II

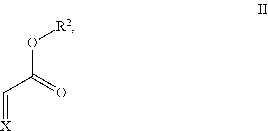

where R² is a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl.

In various embodiments, R¹ comprises an oligomeric or polymeric repeat comprising more than one X, where each X can be the same or different. Such compounds are useful for delivering multiple X, which will become active compound X═O over time. In additional embodiments, R² comprises an oligomeric or polymeric repeat comprising more than one X. Nonlimiting examples of such oligomeric or polymeric repeats include

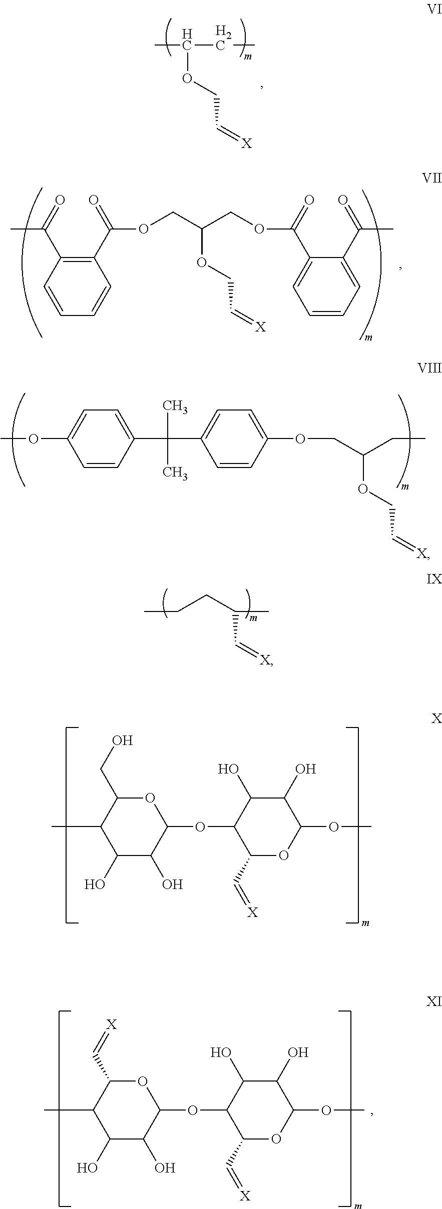

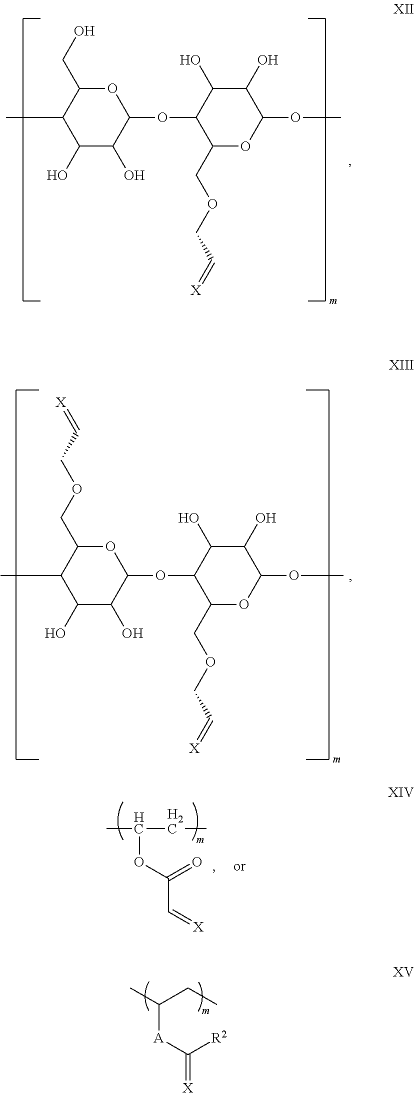

wherein

m is an integer from 2 to 100,000,000,

A is absent or a linking group selected from the group consisting of a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl, and

R² is independently hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl.

In various embodiments, the active compound is a biocide. In some embodiments, the biocide is a pesticide, e.g., a fungicide, an herbicide, an insecticide, an algicide, a molluscicide, a miticide, a repellants, or a rodenticide.

In other embodiments, the biocide is an antimicrobial, e.g., a germicide, an antibiotic, an antibacterial, an antiviral, an antifungal, an antiprotozoal, or an antiparacidal. The antimicrobial can be formulated and utilized as a pharmaceutical or for environmental administration, e.g., inside or outside, and not applied directly to a human or animal. When the antimicrobial is used in the environment, it can be formulated in any form, for example as a paint or a spray, or integrated into a solid material, or coated on the surface of a solid material.

Nonlimiting examples of biocides are (S)-3-anilino-5-methyl-5-phenylimidazolidine-2,4-dione, 1,4-nonyl lactone,1,4-undecanolide, 1-naphthyl-n-methylcarbamate, 2-(1-methylpropyl)phenyl methylcarbamate, 2-(m-chlorophenoxy)propionamide, 2,4-d, 20-hydroxyecdysone, 2-imidazolidone, 2-undecanone, 3′-(trifluoromethyl)acetophenone, 3-hydroxycarbofuran, 3-ketocarbofuran, abamectin, acephate, acetochlor, acetogenins, acetylacetone, acibenzolar-s-methyl, acrinathrin, alachlor, alanycarb, aldicarb, aldicarb-sulfone, aldicarb-sulfoxide, aldoxycarb, allethrin, amicarbazone, amidosulfuron, aminobenzaldehydes, aminocarb, amphotericin b, azadirachtin, azafenidin, azamethiphos, azimsulfuron, azinphos-ethyl, azinphos-methyl, azoxystrobin, barban, benalaxyl, benalaxyl-m, benazolin, benazolin-ethyl, bendiocarb, benodanil, benomyl, benoxacor, bentazon, benzadox, benzaldehydes, benzofenap, benzoin, benzoximate, benzoylureas, bifenazate, bifenthrin, bilanafos, binapacryl, bioallethrin, bioresmethrin, bistrifluron, bixafen, blasticidin s, boscalid, brodaifacoum, bromacil, bromadiolone, bromobutide, bromopropylate, bufencarb, buprofezin, butafenacil, butocarboxim, butoxycarboxim, butoxypropyl ester, caffeine, camphor, capsaicin, captafol, captan, carbaryl, carbendazim, carbetamide, carbofuran, carbofuran-3-keto, carbosulfan, carboxin, carboxine, carpropamid, carvone, chloranil, chlorantraniliprole, chlorbromuron, chlorbufam, chlorfluazuron, chlorimuron ethyl ester, chlorobenzilate, chlorogenic acid, chlorophacinone, chloropropylate, chlorotoluron, chloroxuron, chlorpropham, chlorsulfuron, chlortoluron, chlozolinate, chromafenozide, cinerin, cinnamaldehyde, cinnamyl acetate, cinosulfuron, cis-1,2,3,6-tetrahydrophthalimide, cismethrin, cis-mevinphos, cis-permethrin, citral, citronellal, clethodim, clodinafop-propargyl, cloethocarb, clofencet, clomazone, clomeprop, cloquintocet-mexyl, coumaphos, coumarins, coumatetralyl, crotoxyphos, cyantraniliprole, cyclanilide, cycloheximide, cyclosulfamuron, cycloxydim, cycluron, cyflufenamid, cyfluthrin, cyhalothrin, cymoxanil, cyperethrin, cypermethrin, cyphenothrin, daimuron, daminozide, daptomycin, deet, deguelin, deltamethrin, derris (rotenone), desmedipham, desmethyl-formamido-pirimicarb, dialifos, dibutyl adipate, dichlone, dichlormid, dichlorobenzophenone, diclocymet, diclomezine, dicrotophos, diethofencarb, difenacoum, difenoxuron, difethialone, diflubenzuron, diflufenican, diflufenzopyr, dihydro-5-heptyl-2(3h)-furanone, dihydro-5-pentyl-2(3h)-furanone, dimefluthrin, dimefuron, dimethachlor, dimethenamid, dimethoate, dimethomorph, dimethyl fumarate, dimethyl phthalate, dimetilan, dimoxystrobin, dinobuton, dinocap, dinoterbon, dioxacarb, diphacinone, dipropyl isocinchomeronate, ditalimfos, dithianon, diuron, doramectin, d-phenothrin, drazoxolon, emamectin benzoate, empenthrin, encainide, endrin aldehyde, endrin ketone, eprinomectin, esfenvalerate, ethienocarb, ethiofencarb, ethirimol, ethoxysulfuron, ethyl formate, etobenzanid, famoxadone, fenamidone, fenethacarb, fenfuram, fenobucarb, fenoxacrim, fenoxanil, fenoxaprop ethyl ester, fenoxycarb, fenpropathrin, fenpyroximate, fenuron, fenvalerate, flamprop-isopropyl, flazasulfuron, flocoumafen, flonicamid, fluazifop-p-butyl, fluazolate, fluazuron, flubendiamide, flucycloxuron, flucythrinate, flucytosine, flufenacet, flufenoxuron, flumethrin, flumioxazin, flumipropyn, flumorph, fluometuron, fluopicolide, fluopyram, fluoroacetamide, fluoroimide, fluoroquinolones, flupoxam, flupropacil, flupyrsulfuron, fluquinconazole, fluridone, flurochloridone, fluroxypyr-meptyl, flurtamone, flutolanil, fluxapyroxad, folpet, foramsulfuron, forchlorfenuron, formaldehyde, formetanate, formothion, fosmethilan, fosthiazate, fthalide, furametpyr, furathiocarb, furazolidone, furethrin, furfural, furilazole, glyphosate, glutaraldehyde, griseofulvin, halacrinate, halofenozide, halosafen, haloxyfop methyl ester, hexaflumuron, hexazinone, hexythiazox, hydranal, hydroprene, icaridin, iclosamide, imazamox, imazapic, imazapyr, imazaquin, imazethapyr, imazosulfuron, imiprothrin, inabenfide, indandiones, indanofan, indoxacarb, iprodione, iprovalicarb, isocarbophos, isofenphos, isoprocarb, isoprothiolane, isoproturon, isopyrazam, isotianil, isoxachlortole, ivermectin, jasmolin i,ii, kresoxim-methyl, lactofen, lenacil, linuron, lufenuron, lythidathion, malathion, mandipropamid, mecarbam, mefenacet, mefluidide, mepronil, meptyldinocap, mesotrione, metaflumizone, metalaxyl, metamitron, meta-phthaldialdehyde, metazachlor, methabenzthiazuron, methasulfocarb, methfuroxam, methidathion, methiocarb, methomyl, methoxyfenozide, metobromuron, metofluthrin, metolachlor, metolazone, metolcarb, metominostrobin, metoxadiazone, metoxuron, metrafenone, metribuzin, molinate, monolinuron, monuron, morfamquat, myclozolin, naftalofos, naphthaleneacetamide, naproanilide, naptalam, neburon, neem (azadirachtin), nicosulfuron, nitrobenzaldehydes, nitrofurantoin, norcotinine, norflurazon, novaluron, octanone, octhilinone, ofurace, omethoate, ortho-phthaldialdehyde, orysastrobin, oxadiargyl, oxadiazon, oxadixyl, oxamyl, oxasulfuron, oxaziclomefone, oxolinic acid, oxycarboxin, oxytetracycline, oxythioquinox, para-phthaldialdehyde, pencycuron, penflufen, penthiopyrad, permethrin, phenisopham, phenmedipham, phenothrin, phenserine, phenthoate, phosalone, phosdrin, phosmet, phosphamidon, phosphocarb, phthalaldehydes, phthalamic acid, phthalates, phthaldialdehydes, phthalide, picaridin, pilsicainide, pindone, piperitone, pirimicarb, prallethrin, pretilachlor, prochloraz, procymidone, prohexadione, promecarb, pronamide, propachlor, propamocarb, propaquizafop, propetamphos, propham, propoxur, proquinazid, prosulfuron, pymetrozin, pymetrozine, pyracarbolid, pyraclostrobin, pyrazolynate, pyrazon, pyrazophos, pyresmethrin, pyrethrin, pyrethroids, pyribencarb, pyridaben, pyridaphenthion, pyridate, pyrinuron, pyroquilon, quinacetol, quinoclamine, rafoxanide, ralfinamide, rimsulfuron, rivastigmine, rotenone, safinamide, s-bioallethrin, scilliroside, sedaxane, sethoxydim, siduron, sintofen, sordarin, spinosad, spinosyn d, spiromesifen, spirotetramat, streptomycin, strychnine, sulcotrione, sulfentrazone, tebufenozide, tebufenpyrad, tebuthiuron, tecloftalam, teflubenzuron, telithromycin, tepraloxydim, terallethrin, terbacil, terbucarb, terephthalaldehyde, tetramethrin, tetranortriterpenoid, thenylchlor, thiacloprid-amide, thidiazimin, thidiazuron, thifluzamide, thiofanox, tiadinil, tocainide, tolfenpyrad, tolperisone, tralkoxydim, tralomethrin, transfluthrin, trans-mevinphos, trans-permethrin, triadimefon, triasulfuron, triazamate, triazofenamide, trichloroisocyanuric acid, trifloxysulfuron, triflumuron, triforin, triforine, trimethacarb, trinexapac-ethyl, valifenalate, vamidothion, vinclozolin, warfarin, ylachlor, and zoxamide.

Scheme 3 shows the production of the antibacterial compound glutaraldehyde from a noncyclic and cyclic polymer.

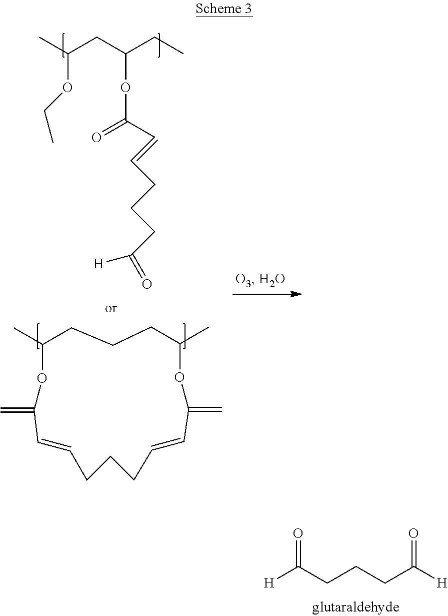

The effectiveness of these compounds can be tested by any means known in the art. In some embodiments, an inactive antibacterial compound such as those shown in Scheme 3, can be tested for the release of the activated compound by spotting the inactive compound on a bacterial lawn, e.g., in a petri dish, in the presence and absence of ozone, where, with an inactive compound that effectively reacts with ozone to release the active antibacterial compound, the bacteria around the ozone reacting compound are killed but the bacteria around the compound where ozone is absent will not be killed.

In further embodiments, the active compound is a nontoxic useful compound, such as a cosmetic or a fertilizer, e.g., urea. An inactive compound that provides a fertilizer such as urea after exposure to ozone would provide a slow release fertilizer, which would require fewer applications, and potentially avoid fertilizer runoff, providing less fertilizer loss and environmental contamination, than standard fertilizer. The degradation of ozone during the activation of the fertilizer could also provide protection from ozone damage to the plants.

In these embodiments, the fertilizer can be released from an inactive compound that is a small molecule or polymer. The inactive compound can also be cationic, which would be held in soils that have significant cation exchange capacity, thus further avoiding loss of fertilizer by runoff.

In additional embodiments, the active compound is a pharmaceutical. The pharmaceutical composition is administered locally and/or systemically. As used herein, the term “local administration” is meant to describe the administration of a pharmaceutical composition of the invention to a specific tissue or area of the body with minimal dissemination of the composition to surrounding tissues or areas. Locally administered pharmaceutical compositions are not detectable in the general blood stream when sampled at a site not immediate adjacent or subjacent to the site of administration.

As used herein the term “systemic administration” is meant to describe in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. The pharmaceutical can be used anywhere ozone is available to react with the inactive compound to form the active compound. Examples include the bloodstream, GI tract, oral administration, intramuscular, intraperitoneal, intranasal, etc Further, the pharmaceutical can be used to treat any disease, e.g., cancer, cardiovascular diseases, inflammatory diseases, etc.

The pharmaceutical is formulated such that an effective dose of the active compound is provided after administration and exposure to ozone at the site of activation. Thus, the administration of an effective dose of a particular active compound would require a greater dose of the inactive compound if administered to a site that has a low level of ozone (e.g., the blood stream) than if administered to a site that has a higher level of ozone (e.g., the lungs or the skin). Alternatively, the ozone can be provided in the excipient in which the inactive compound is formulated.

As discussed above, activation is most rapid where the inactive compound is exposed to a relatively high concentration of ozone, e.g., the air. Thus, while the compounds of the present invention could be formulated to be administered systemically, pharmaceutical treatments that provide for exposure of the active compound to the air can provide effective release of the active compound over time, such that administration of the inactive compound to provide a steady dosage of the active compound can be less frequent than the administration of the active compound.

Thus, in some of these embodiments, the inactive compound is inhaled or applied to the skin or another body part that is exposed to air. Thus, the pharmaceutical can effectively be a treatment for a lung disease or disorder, e.g., asthma or COPD, where the inactive compound is inhaled. The pharmaceutical can also be a treatment for a skin disease or disorder or wound, where the inactive compound is applied to the skin. Additionally, the pharmaceutical can be a treatment for an eye disease or disorder, where the inactive compound is applied to the eye.

In additional embodiments, the pharmaceutical is a nutrient, an antibiotic, an antifungal, an antiviral or an antiparasitic, as described above.

Although the concentration of atmospheric ozone is much higher than in bodily tissues that are not exposed to the atmosphere, ozone is nonetheless present in internal tissues, for example in inflammed tissues (see, e.g., EP1929313; US 20050085557). Additionally, oxonolysis products are formed in tissues without ozone, for example through the myeloperoxidase-H₂O₂-chloride system (Tomono et al., 2009, Biochem. Biophys. Res. Comm. 383:222-227). Since myeloperoxidase is particularly abundant in neutrophil granulocytes, a white blood cell, ozonolysis reactions occur in the bloodstream. Myeloperoxidase is also particularly elevated in inflammatory tissue and in diseased cardiovascular tissue (Brennan et al., 2003, New Eng. J. Med. 349:1595-1604).

Since ozonolysis reactions occur in tissues that are not exposed to atmospheric ozone, the above-described pharmaceutical compounds that are activated by ozone can be utilized in those tissues. Thus, the present invention also provides a method of treating a disease or condition in a subject. The method comprises administering the above-described pharmaceutical compound to the subject at a site that is not exposed to atmospheric ozone. In some embodiments, a myeloperoxidase is present at the site. In other embodiments, a neutrophil is present at the site. In further embodiments another white blood cell is present that provides an enzyme, such as myeloperoxidase, to induce an ozonolysis reaction, with or without the presence of ozone. Non-limiting examples of such cells include macrophages, monocytes, lymphocytes, basophils, and eosinophils.

In additional embodiments, the site is the bloodstream of the subject. As such, the pharmaceutical compounds, when administered into the bloodstream, would provide a slow-release production of the activated pharmaceutical compound as blood ozonolysis, for example those mediated by myeloperoxidase, slowly activates the pharmaceutical compound.

Since myeloperoxidase is particularly abundant in inflamed tissues, ozonolysis reactions would be expected to be particularly active in those inflamed tissues and diseased cardiovascular tissues. Thus, anti-inflammatory and cardiovascular (e.g., used to treat or prevent atherosclerosis) active pharmaceutical compounds are particularly useful in these embodiments.

Further, the mitochondria can be targeted, e.g., by incorporating triphenylphosphonium cation, and other means known in the art, for example by creating positive charges, delocalized cations, and cations that partake in resonance structures.

Pharmaceutically acceptable carriers for formulation of the inactive compound may be covalently or non-covalently bound, admixed, encapsulated, conjugated, operably-linked, or otherwise associated with the inactive compound such that the excipient increases the cellular uptake, stability, solubility, half-life, binding efficacy, specificity, targeting, distribution, absorption, or renal clearance of the inactive or active compound. Alternatively, or in addition, the pharmaceutically acceptable carrier increases or decreases the immunogenicity of the inactive or active compound.

Alternatively, or in addition, pharmaceutically acceptable carriers are salts (for example, acid addition salts, e.g., salts of hydrochloric, hydrobromic, acetic acid, and benzene sulfonic acid), esters, salts of such esters, or any other compound which, upon administration to a subject, are capable of providing (directly or indirectly) the inactive or active compounds of the invention. Pharmaceutically acceptable carriers are alternatively or additionally diluents, excipients, adjuvants, emulsifiers, buffers, stabilizers, and/or preservatives.

Pharmaceutically acceptable carriers of the invention include delivery systems/mechanisms that increase uptake of the inactive compound by targeted cells. For example, pharmaceutically acceptable carriers of the invention are viruses, recombinant viruses, engineered viruses, viral particles, replication-deficient viruses, liposomes, cationic lipids, anionic lipids, cationic polymers, polymers, hydrogels, micro- or nano-capsules (biodegradable), micropheres (optionally bioadhesive), cyclodextrins, plasmids, mammalian expression vectors, proteinaceous vectors, or any combination of the preceding elements (see, O'Hare and Normand, International PCT Publication No. WO 00/53722; U.S. Patent Publication 2008/0076701). Moreover, pharmaceutically acceptable carriers that increase cellular uptake can be modified with cell-specific proteins or other elements such as receptors, ligands, antibodies to specifically target cellular uptake to a chosen cell type.

In another aspect of the invention, compositions are first introduced into a cell or cell population that is subsequently administered to a subject. In some embodiments, the inactive compound is delivered intracellularly, e.g., in cells of a target tissue such as lung, or in inflamed tissues. Included within the invention are compositions and methods for delivery of the inactive compound and/or composition by removing cells of a subject, delivering the isolated inactive compound or composition to the removed cells, and reintroducing the cells into a subject. In some embodiments, a miRNA and/or miRNA inhibitor molecule is combined with a cationic lipid or transfection material such as LIPOFECTAMINE (Invitrogen).

In one aspect, the active compounds are prepared with pharmaceutically acceptable carriers that will protect inactive or active compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Examples of materials which can form hydrogels include polylactic acid, polyglycolic acid, PLGA polymers, alginates and alginate derivatives, gelatin, collagen, agarose, natural and synthetic polysaccharides, polyamino acids such as polypeptides particularly poly(lysine), polyesters such as polyhydroxybutyrate and poly-epsilon.-caprolactone, polyanhydrides; polyphosphazines, poly(vinyl alcohols), poly(alkylene oxides) particularly poly(ethylene oxides), poly(allylamines) (PAM), poly(acrylates), modified styrene polymers such as poly(4-aminomethylstyrene), pluronic polyols, polyoxamers, poly(uronic acids), poly(vinylpyrrolidone) and copolymers of the above, including graft copolymers.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Pharmaceutically acceptable carriers are cationic lipids that are bound or associated with miRNA and/or miRNA inhibitor. Alternatively, or in addition, the inactive compounds are encapsulated or surrounded in cationic lipids, e.g. lipsosomes, for in vivo delivery. Exemplary cationic lipids include, but are not limited to, N41-(2,3-dioleoyloxy)propyliN,N,N-trimethylammonium chloride (DOTMA); 1,2-bis(oleoyloxy)-3-3-(trimethylammonium)propane (DOTAP), 1,2-bis(dimyrstoyloxy)-3-3-(trimethylammonia)propane (DMTAP); 1,2-dimyristyloxyprop y1-3-dimethylhydroxyethylammonium bromide (DMRIE); dimethyldioctadecylammonium bromide (DDAB); 3-(N—(N′,N′-dimethylaminoethane)carbamoyl)cholesterol (DC-Chol); 3β-[N′,N′-diguanidinoethyl-aminoethane)carbamoyl cholesterol (BGTC); 2-(2-(3-(bis(3-aminopropyl)amino)propylamino)acetamido)-N,N-ditetradecyla-cetamide (RPR209120); pharmaceutically acceptable salts thereof, and mixtures thereof. Further exemplary cationic lipids include, but are not limited to, 1,2-dialkenoyl-sn-glycero-3-ethylphosphocholines (EPCs), such as 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, pharmaceutically acceptable salts thereof, and mixtures thereof.

Exemplary polycationic lipids include, but are not limited to, tetramethyltetrapalmitoyl spermine (TMTPS), tetramethyltetraoleyl spermine (TMTOS), tetramethlytetralauryl spermine (TMTLS), tetramethyltetramyristyl spermine (TMTMS), tetramethyldioleyl spermine (TMDOS), pharmaceutically acceptable salts thereof, and mixtures thereof. Further examplary polycationic lipids include, but are not limited to, 2,5-bis(3-aminopropylamino)-N-(2-(dioctadecylamino)-2-oxoethyl)pentanamid-e (DOGS); 2,5-bis(3-aminopropylamino)-N-(2-(di(Z)-octadeca-9-dienylamino)-2-oxoethyl)pentanamide (DOGS-9-en); 2,5-bis(3-aminopropylamino)-N-(2-(di(9Z,12Z)-octadeca-9,12-dienylamino)-2-oxoethyl)pentanamide (DLinGS); 3-beta-(N4-(N1, N8-dicarbobenzoxyspermidine)carbamoyl)chole-sterol (GL-67); (9Z,9yZ)-2-(2,5-bis(3-aminopropylamino)pentanamido)propane-1,3-diyl-dioct-adec-9-enoate (DOSPER); 2,3-dioleyloxy-N-[2(sperminecarboxamido)ethyl]-N,N-dimethyl-1-propanamini-urn trifluoro-acetate (DOSPA); pharmaceutically acceptable salts thereof, and mixtures thereof.

Examples of cationic lipids are described in U.S. Pat. Nos. 4,897,355; 5,279,833; 6,733,777; 6,376,248; 5,736,392; 5,334,761; 5,459,127; 2005/0064595; U.S. Pat. Nos. 5,208,036; 5,264,618; 5,279,833; 5,283,185; 5,753,613; and 5,785,992; each of which is incorporated herein in its entirety.

Pharmaceutically acceptable carriers of the invention also include non-cationic lipids, such as neutral, zwitterionic, and anionic lipids. Examplary non-cationic lipids include, but are not limited to, 1,2-Dilauroyl-sn-glycerol (DLG); 1,2-Dimyristoyl-snglycerol (DMG); 1,2-Dipalmitoyl-sn-glycerol (DPG); 1,2-Distearoyl-sn-glycerol (DS G); 1,2-Dilauroyl-sn-glycero-3-phosphatidic acid (sodium salt; DLPA); 1,2-Dimyristoyl-snglycero-3-phosphatidic acid (sodium salt; DMPA); 1,2-Dipalmitoyl-sn-glycero-3-phosphatidic acid (sodium salt; DPPA); 1,2-Distearoyl-sn-glycero-3-phosphatidic acid (sodium salt; DSPA); 1,2-Diarachidoyl-sn-glycero-3-phosphocholine (DAPC); 1,2-Dilauroyl-sn-glycero-3-phosphocholine (DLPC); 1,2-Dimyristoyl-sn-glycero-3-phosphocholine (DMPC); 1,2-Dipalmitoyl-sn-glycero-O-ethyl-3-phosphocholine (chloride or triflate; DPePC); 1,2-Dipalmitoyl-sn-glycero-3-phosphocholine (DPPC); 1,2-Distearoyl-sn-glycero-3-phosphocholine (DSPC); 1,2-Dilauroyl-sn-glycero-3-phosphoethanolamine (DLPE); 1,2-Dimyristoyl-sn-glycero-3-phosphoethanolamine (DMPE); 1,2-Dipalmitoyl-sn-glycero-3-phosphoethanolamine (DPPE); 1,2-Distearoylsn-glycero-3-phosphoethanolamine (DSPE); 1,2-Dilauroyl-sn-glycero-3-phosphoglycerol (sodium salt; DLPG); 1,2-Dimyristoyl-sn-glycero-3-phosphoglycerol (sodium salt; DMPG); 1,2-Dimyristoyl-sn-glycero-3-phospho-sn-1-glycerol (ammonium salt; DMP-sn1-G); 1,2-Dipalmitoyl-sn-glycero-3-phosphoglycerol (sodium salt; DPPG); 1,2-Distearoyl-sn-glycero-3-phosphoglycero (sodium salt; DSPG); 1,2-Distearoyl-snglycero-3-phospho-sn-1-glycerol (sodium salt; DSP-sn-1-G); 1,2-Dipalmitoyl-snglycero-3-phospho-L-serine (sodium salt; DPP S); 1-Palmitoyl-2-linoleoyl-sn-glycero-3-phosphocholine (PLinoPC); 1-Palmitoyl-2-oleoyl-sn-glycero-3-phosphocholine (POPC); 1-Palmitoyl-2-oleoyl-sn-glycero-3-phosphoglycerol (sodium salt; POPG); 1-Palmitoyl-2-oleoyl-sn-glycero-3-phosphoglycerol (sodium salt; POPG); 1-Palmitoyl-2-oleoyl-snglycero-3-phosphoglycerol (ammonium salt; POPG); 1-Palmitoyl-2-4-o-sn-glycero-3-phosphocholine (P-lyso-PC); 1-Stearoyl-2-lyso-sn-glycero-3-phosphocholine (S-lysoPC); and mixtures thereof. Further exemplary non-cationic lipids include, but are not limited to, polymeric compounds and polymer-lipid conjugates or polymeric lipids, such as pegylated lipids, including polyethyleneglycols, N-(Carbonylmethoxypolyethyleneglycol-2000)-1,2-dimyristoyl-sn-glycero-3-phosphoethanolamine (sodium salt; DMPE-MPEG-2000); N-(Carbonyl-methoxypolyethyleneglycol-5000)-1,2-dimyristoyl-sn-glycero-3-phosphoethanolamine (sodium salt; DMPE-MPEG-5000); N(Carbonyl-methoxypolyethyleneglycol 2000)-1,2-dipalmitoyl-sn-glycero-3-phosphoethanolamine (sodium salt; DPPE-MPEG-2000); N-(Carbonyl-methoxypolyethyleneglycol 5000)-1,2-dipalmitoyl-sn-glycero-3-phosphoethanolamine (sodium salt; DPPE-MPEG-5000); N-(Carbonyl-methoxypolyethyleneglycol 750)-1,2-distearoyl-sn-glycero-3-phosphoethanolamine (sodium salt; DSPE-MPEG-750); N(Carbonyl-methoxypolyethyleneglycol 2000)-1,2-distearoyl-sn-glycero-3-phosphoethanolamine (sodium salt; DSPE-MPEG-2000); N-(Carbonylmethoxypolyethyleneglycol 5000)-1,2-distearoyl-sn-glycero-3-phosphoethanolamine (sodium salt; DSPE-MPEG-5000); sodium cholesteryl sulfate (SCS); pharmaceutically acceptable salts thereof, and mixtures thereof. Examples of non-cationic lipids include, but are not limited to, dioleoylphosphatidylethanolamine (DOPE), diphytanoylphosphatidylethanolamine (DPhPE), 1,2-Dioleoyl-sn-Glycero-3-Phosphocholine (DOPC), 1,2-Diphytanoyl-sn-Glycero-3-Phosphocholine (DPhPC), cholesterol, and mixtures thereof.

Pharmaceutically-acceptable carriers of the invention further include anionic lipids. Exemplary anionic lipids include, but are not limited to, phosphatidylserine, phosphatidic acid, phosphatidylcholine, platelet-activation factor (PAF), phosphatidylethanolamine, phosphatidyl-DL-glycerol, phosphatidylinositol, phosphatidylinositol (pi(4)p, pi(4,5)p2), cardiolipin (sodium salt), lysophosphatides, hydrogenated phospholipids, sphingoplipids, gangliosides, phytosphingosine, sphinganines, pharmaceutically acceptable salts thereof, and mixtures thereof.

Supplemental or complementary methods for delivery of nucleic acid molecules for use herein are described, e.g., in Akhtar, et al., Trends Cell Bio. 2:139, 1992; Delivery Strategies for Antisense Oligonucleotide Therapeutics, ed. Akhtar, 1995; Maurer, et al., Mol. Membr. Biol. 16:129-140, 1999; Hofland and Huang, Handb. Exp. Pharmacol. 137:165-192, 1999; and Lee, et al., ACS Symp. Ser. 752:184-192, 2000. Sullivan, et al., International PCT Publication No. WO 94/02595, further describes general methods for delivery of enzymatic nucleic acid molecules. These protocols can be utilized to supplement or complement delivery of virtually any inactive compound of the invention.

In various embodiments, the pharmaceutical is useful for treatment of a lung, eye, skin, nasal, oral, scalp, or nail disease or disorder.

In certain embodiments, the pharmaceutical is an oligopeptide, a polypeptide, or a steroid, for example estrone, cortisol, corticosterone, aldosterone, progesterone, testosterone, or dihydrotestosterone.

The pharmaceutical can also be a nutrient, e.g., vitamin B12, or any other nutrient that has a carbonyl group.

Nonlimiting examples of pharmaceuticals include β2-Adrenergic Receptor Agonists, (+)-6-Aminopenicillanic acid, (S)-(+)-Camptothecin, 10-Deacetylbaccatin III, 17α-Hydroxy Pregnenolone, 17α-Hydroxy Progesterone, 5-Azacytidine, 6-OHM, 7-Aminocephalosporanic acid, 7-Aminodesacetoxycephalosporanic acid, 8-Chlorotheophylline, 8-Cyclopentyl-1,3-dimethylxanthine, 8-Phenyltheophylline, A-349,821, Abarelix (Plenaxis), Abecarnil, Abelcet (Amphotericin B), Abilify (Aripiprazole), Abraxane, Acaprazine, Acebutolol (Sectral), Aceon (Perindopril Erbumine), Acepromazine, Acetadote (Acetylcysteine), Acetaminophen (Tylenol), Acetazolamide, Acetominophen, Acetylcholine Chloride (Miochol-E), Acetylcysteinamide, Acetyldihydrocodeine, Acetylsalicylic acid (Aspirin), Aciclovir, Acitretin (Soriatane), Aclidinium, Aclovate (Alclometasone Dipropionate), Acrivastine, Acticlate (Doxycycline Hyclate), Actinomycins, Actinonin, Acular (Ketorolac Tromethamine), Acycloguanosine, Acyclovir (Zovirax), Acylampicllins, Adalat CC (Nifedipine), Adapalene, Adcetris (Brentuximab Vedotin), Adcirca (Tadalafil), Adempas (Riociguat), Ado-trastuzumab Emtansine (Kadcyla), Adriamycin PFS (Doxorubicin hydrochloride), Advair Diskus (Fluticasone Propionate), Afatinib (Gilotrif), Afinitor (Everolimus), Aflibercept (Eylea), Afloqualone, Aggrastat, Agomelatine, Agrylin (Anagrelide), AH-7921, Ak-Fluor (Fluorescein), Alamethicin, Albendazole, Albiglutide (Tanzeum), Alcaftadine, Alclometasone Dipropionate (Aclovate), Aldesleukin (Proleukin), Aldomet (Methyldopa), Aldoril (Methyldopa-Hydrochlorothiazide), Aldosterone, Aldurazyme (Laronidase), Alemtuzumab (Campath, Lemtrada), Alfadolone, Alfaxalone, Alfenta (Alfentanil), Alfuzosin HCl (Uroxatral), Alglucerase (Ceredase), Alglucosidase Alfa (Lumizyme, Myozyme), Alimta (Pemetrexed), Alinia (Nitazoxanide), Aliskiren (Tekturna), Alitretinoin (Panretin), Alizapride, Alkaloids, Alkeran (Melphalan), Allegra (Fexofenadine HCl), Alli (Orlistat), Allobarbital, Allopregnanolone, Allopurinol (Zyloprim), Alnespirone, Alocril (Nedocromil), Alogliptin (Nesina), Alomide (Lodoxamide Tromethamine), Aloprim (Allopurinol Sodium), Alosetron Hydrochloride (Lotronex), Aloxi (Palonosetron HCl), Alpha (Prolastin), Alpha-Galactosidase, Alphanate (Antihemophilic Factor), Alphenal, Alpidem, Alprostadil, Alrex (Loteprednol Etabonate), Altabax (Retapamulin), Altace (Ramipril), Alteplase (Activase), Altocor (Lovastatin), Alvesco (Ciclesonide), Alvimopan (Entereg), Amaryl (Glimepiride), Ambenonium, Ambien, Ambisome (Amphotericin B), Ambrisentan (Volibris), Amcinonide, Americaine (Benzocaine), A-Methapred (Methylprednisolone Sodium Succinate), Amevive (Alefacept), Amicar (Aminocaproic Acid), Amidotrizoate, Amikacins, Amiloride, Amineptine, Amino Acids, Aminocaproic Acid (Amicar), Aminocoumarins, Aminoglutethimide (Cytadren), Aminoglycosides, Aminohippurate (Aminohippurate Sodium), Aminolevulinic Acid (Levulan Kerastick), Aminopenicillins, Aminosalicylic Acids, Amiodarone, Amisulpride, Amitiza (Lubiprostone), Amlexanox (Aphthasol), Amlopidine, Amobarbital, Amoxicillin, Amphenicols, Amphotericin B, Ampicillin, Amprenavir (Agenerase), Amytal Sodium (Amobarbital Sodium), Anabolic Steroids, Anadrol-50 (Oxymetholone), Anagrelide (Agrylin), Anakinra (Kineret), Ancobon (Flucytosine), Androgens, Androstanediols, Androstanes, Androstenediols, Androstenediones, Anectine (Succinylcholine Chloride), Angeliq (Drospirenone), Angiomax (Bivalirudin), Anhydroerythromycin A, Anidulafungin, Anisindione (Miradon), Anisomycin, Ansaid (Flurbiprofen), Ansamycins, Antara (Fenofibrate), Anthracyclines, Anthralin (Dritho-Scalp), Antibodies, Antihemophilic Factor (Alphanate, Bioclate, Koate, Monoclate, Refacto, Xyntha, Helixate FS), Antimycin A, Antimycin A2, Antimycins, Antipain, Antipyrine, Antithrombin (Thrombate), Antrafenine, Anturane (Sulfinpyrazone), Anturol (Oxybutynin), Anusols, Anzemet (Dolasetron Mesylate), ApexiCon E (Diflorasone Diacetate), Aphidicolin, Aphrodyne (Yohimbine), Aphthasol (Amlexanox), Apidra (Insulin Glulisine [rDNA origin]), Apixaban (Eliquis), Aplenzin (Bupropion Hydrobromide), Apremilast (Otezla), Aprepitant, Apriso (Mesalamine), Aprobarbital, Aprotinin (Trasylol), Aptivus (Tipranavir), Aranesp (Darbepoetin Alfa), Arava (Leflunomide), Arbekacin, Arcalyst (Rilonacept), Arcapta Neohaler (Indacaterol), Arestin (Minocycline Hydrochloride Microspheres), Arformoterol, Argatroban, Aricept (Donepezil Hydrochloride), Aripiprazole, Aristocort (Triamcinolone Diacetate), Armodafinil (Nuvigil), Aromasin (Exemestane), Artesunate, Articaine, Arzerra (Ofatumumab), Asacol (Mesalamine), Ascochlorin, Ascomycin, Ascorbic acid, Asmanex Twisthaler (Mometasone Furoate), Asparaginase, Aspirine, Astagraf XL (Tacrolimus), Astelin (Azelastine Hydrochloride), Astromicin, Atacand (Candesartan Cilexetil), Ataluren, Atazanavir, Atenolol, Atevirdine, Atgam (Lymphocyte immune globulin), Ativan (Lorazepam), Atorvastatin, Atovaquone (Wellvone), Atracurium, Atralin (Tretinoin), Atridox (Doxycycline Hyclate), Atromid-S(Clofibrate), Atropen (Atropine), Atrovent (Ipratropium Bromide), Atryn, Aubagio (Teriflunomide), Augmentin (Amoxicillin Clavulanate), Auranofin (Ridaura), Aureomycin, AV-101, Avage (Tazarotene), Avalide (Irbesartan-Hydrochlorothiazide), Avanafil (Stendra), Avapro (Irbesartan), Avastin (Bevacizumab), Aveed (Testosterone Undecanoate), Avelox (Moxifloxacin), Avibactam, Avilamycin, Avita (Tretinoin), Avodart (Dutasteride), Avonex (Interferon beta-1a), Avoparcin, Axilsartan Medoxomil, Axitinib (Inlyta), Aygestin (Norethindrone), Azacitidine (Vidaza), Azactam (Aztreonam), Azaperone, Azapirones, Azasite (Azithromycin), Azaspirodecanedione, Azelaic Acid (Finacea), Azelastine, Azidamfenicol, Azilsartan medoxomil (Edarbi), Azithromycin, Azlocillin, Azmacort (Triamcinolone Acetonide), Aztreonam, Azulfidine (Sulfasalazine), BAAM, Bacampicilin, Bacitracin, Baclofen (Kemstro), Bactenecin, Bactroban (Mupirocin Calcium), Bafilomycin A1, Bafilomycin B1, Balsalazide (Colazal), Bambermycins (Flavomycin), Banzel (Rufinamide), Baraclude (Entecavir), Barbexaclone, Barbital, Barbiturates, Barbituric Acid, Basiliximab (Simulect), Batoprazine, Bayer (Aspirin), Becaplermin (Regranex), Beclamide, Beclometasone, Befiradol, Befloxatone, Befunolol, Belatacept (Nulojix), Beleodaq (Belinostat), Belimumab (Benlysta), Belsomra (Suvorexant), Benazepril, Bendamustine, Benlysta (Belimumab), Benmoxin, Benoxinate, Benperidol, Bentazepam, Bentyl (Dicyclomine), Benzamycin (Erythromycin), Benzathine benzylpenicillin, Benzathine penicillin, Benznidazole, Benzocaine, Benzodiazepines, Benzoic Acid, Benzonatate, Benzylbutylbarbiturate, Benzylpenicillin, Bepotastine, Beractant (Survanta), Bergapten, Besifloxacin (Besivance), Bestatin, Beta Lactams, Betadine, Beta-Galactosidase, Betagan (Levobunolol), Betamethasones, Bethanechol, Betulinic Acid, Bevacizumab, Bexarotene (Targretin), Biaxin (Clarithromycin), Bicalutamide, BiCNU (Carmustine), Bicuculline, Bifeprunox, Bilastine, Biltricide (Praziquantel), Binospirone, Bioclate (Antihemophilic Factor), Bionect (Hyaluronic acid sodium salt), Bisacodyl, Bismuth Subsalicylate, Bivalirudin (Angiomax), Blasticidin S, Blenoxane (Bleomycin), Blinatumomab (Blincyto), Bloxiverz (Neostigmine), Boceprevir, Bortezomib (Velcade), Botox, Brallobarbital, Bravelle (Urofollitropin), Brefeldin A, Brentuximab Vedotin (Adcetris), Bretazenil, Brevibloc (Esmolol), Brevital (Methohexital), Brexpiprazole, Brivaracetam, Bromazepam, Bromday (Bromfenac), Bromocriptine, Bromopride, Bromoxanide, Brovana (Arformoterol Tartrate), Budeprion, Budesonide, Bumetanide (Bumex), Buphenyl (Sodium Phenylbutyrate), Bupivacaine, Bupropion, Buspirone, Butabarbital, Butalbital, Butamben, Butyrophenones, BW373U86, Bydureon (Exenatide), Cabergoline, Cabozantinib (Cometriq), Caerulomycin A, Caffeine, Calcipotriene, Calcitonin, Calcium Ionophore A23187, Calcium Ionophore III, Cambia (Diclofenac), Campath (Alemtuzumab), Campral, Camptosar (Irinotecan), Canakinumab, Canasa (Mesalamine), Cancidas (Caspofungin), Candesartan, Cantil (Mepenzolate), Capastat Sulfate (Capreomycin), Capecitabine (Xeloda), Capobenic Acid, Capoten (Captopril), Capreomycin, Capsaicin (Qutenza), Captopril (Capoten), Carac (Fluorouracil), Carbacephems, Carbachol (Miostat), Carbaglu (Carglumic Acid), Carbamates, Carbamazepines, Carbapenems, Carbenicillin, Carbidopa (Lodosyn), Carbocaine (Mepivacaine), Carbomycin, Carboplatin (Paraplatin), Carboprost, Carboxypenicillins, Cardizem (Diltiazem), Cardura (Doxazosin Mesylate), Carfentanil, Carfilzomib (Kyprolis), Cariprazine, Carisoprodol, Carmustine (BiCNU), Carnitor (Levocarnitine), Caroxazone, Carteolol, Cartia XT (Diltiazem), Casodex (Bicalutamide), Casopitant, Caspofungin Acetate (Cancidas), Cataflam (Diclofenac), Cathelicidins, Cathinones, Caverject (Alprostadil), Cayston (Aztreonam), Ceclor (Cefaclor), Cecropins, Cedax (Ceftibuten), CeeNU (Lomustine), Cefaclor, Cefadroxil, Cefalexin, Cefalotin, Cefamandole, Cefapirin, Cefazolin, Cefdinir (Omnicef), Cefditoren Pivoxil (Spectracef), Cefepime, Cefixime (Suprax), Cefizox (Ceftizoxime), Cefmetazole, Cefobid (Cefoperazone), Cefotan (Cefotetan), Cefotaxime, Cefoxitin (Mefoxin), Cefpodoxime, Cefprozil (Cefzil), Cefsulodin, Ceftaroline Fosamil, Ceftazidime (Ceptaz), Ceftibuten, Ceftin (Cefuroxime Axetil), Ceftizoxime, Ceftobiprole, Ceftolozane, Ceftriaxone, Cefuroxime (Zinacef), Cefzil (Cefprozil), CellCept (Mycophenolate Mofetil), Celontin (Methsuximide), Cenestin, Centany (mupirocin), Cephalexin (Keflex), Cephalomannine, Cephalosporins, Cephalothin, Cephamycins, Cephems, Cephradine, Ceptaz (Ceftazidime), CERC-301, CERC-501, Cerdelga (Eiglustat), Cerebyx (Fosphenytoin), Cerezyme (Imiglucerase), Certolizumab Pegol, Cerubidine (Daunorubicin), Cerulenin, Cervidil (Dinoprostone), Cesamet (Nabilone), Cethromycin, Cetirizine, Cetraxal (Ciprofloxacin Otic), Cetrorelix (Cetrotide), Cetuximab (Erbitux), CGS-20625, CGS-9896, Chibroxin (Norfloxacin), Chlorambucil, Chloramphenicols, Chlorazepate, Chloroprocaine (Nesacaine), Chloroptic (Chloramphenicol), Chlorpropamide (Diabinese), Chlortetracycline, Chlorthalidone (Thalitone), Chlorzoxazone, Cholbam (Cholic Acid), Chromomycins, Cicatrizants, Ciclesonide, Ciclopirox, Ciclosporin, Cidofovir (Vistide), Ciladopa, Cilastatin, Cilostazol (Pletal), Cimoxatone, Cimzia (Certolizumab), Cinchocaine, Cindamycin, Cinitapride, Cinnamycin, Cinobac (Cinoxacin), Ciprofloxacin, Ciproxifan, Cisapride, Cisatracurium Besilate (Nimbex), Citric Acid, Civetone, Claforan (Cefotaxime), Clarithromycin, Claritin (Loratadine), Clavulanate (Augmentin), Clavulanic Acid, Clebopride, Clevidipine Butyrate (Cleviprex), Clexane, Clidinium, Clindamycin, Clinoril (Sulindac), Clioxanide, Clobazam, Clobetasols, Clobetasones, Clobex, Clocortolone (Cloderm), Clofibrate, Clomocycline, Clonazepam, Clopidogrel Bisulfate (Plavix), Clorazepate Dipotassium (Tranxene), Cloroqualone, Clotiazepam, Cloxacillin, Cobicistat, Cobicistat (Tybost), Colazal (Balsalazide), Colchicine, Colistin, Colominic acid, Combivir, Cometriq (Cabozantinib), Comtan (Entacapone), Concanamycin A, Concerta (Methylphenidate), Condylox (Podofilox), Copaxone (Glatiramer Acetate), Copegus (Ribavirin), Cordarone (Amiodarone), Cordran (Clurandrenolide), Corlanor (Ivabradine), Cormax (Clobetasol Propionate), Cortaid (Hydrocortisone), Corticosteroids, Corticosterone, Cortisol, Cortisone, Cosentyx (Secukinumab), Cosmegen (Dactinomycin), Cosyntropin, Coumadin (Warfarin), Coumarins, Coumermycin A1, Creatine, Creatinine, Crestor (Rosuvastatin Calcium), Crinone (Progesterone), Crixivan (Indinavir Sulfate), Crolom, Cromoglicic Acid, Crotamiton, CSP-2503, Cubicin (Daptomycin), Cuprimine (Penicillamine), Curosurf (Poractant Alfa), Cuvposa (Glycopyrrolate), Cyanocobalamin, Cyclic Lipopeptides, Cyclodextrins, Cycloheximide, Cyclopyrrolones, Cycloserine, Cycloset (Bromocriptine Mesylate), Cyclosporins, Cyklokapron (Tranexamic Acid), Cylert (Pemoline), Cytadren (Aminoglutethimide), Cytarabine, Cytochalasins, Cytomel (Liothyronine), Cytosar-U, Cytotec (Misoprostol), Cytovene (Ganciclovir), D. H. E. 45 (Dihydroergotamine), Dabigatran Etexilate Mesylate (Pradaxa), Dacarbazine, Daclatasvir, Daclizumab (Zenapax), Dacogen (Decitabine), Dactinomycin, Dalbavancin, Dalfopristin, Daliresp (Roflumilast), Dalmane (Flurazepam), Dalteparin (Fragmin), Dantrium (Dantrolene Sodium), Daptomycin, Darbepoetin Alfa (Aranesp), Darifenacin (Enablex), Darunavir (Prezista), Dasabuvir, Dasatinib, Daunorubicin (Cerubidine), Daypro, Dazopride, DDAVP, Decadron (Dexamethasone), Decitabine, Declomycin (Demeclocycline HCl), Defensins, Deferiprone (Ferriprox), Deferoxamine (Desferal), Degarelix (Firmagon), Dehydroepidandrosterone, Delavirdine, Delzicol (Mesalamine), Demadex (Torsemide), Demerol (Meperidine), Denavir (Penciclovir), Deogestrel, Deoxycorticosterone, Depo Medrol (Methylprednisolone Acetate), DepoCyt (Cytarabine Liposome), Depo-Provera (Medroxyprogesterone), Desferal (Deferoxamine), Desirudin (Iprivask), Desmopressin, Desonate (Desonide), Desoximetasone (Topicort), Dexamethasone, Dexmethylphenidate Hydrochloride (Focalin), Dexrazoxane (Zinecard), Dextropropoxyphene, Dht (Dihydrotachysterol), DiaBeta (Glyburide), Diabinese (Chlorpropamide), Diazepam, Dibenzepin, Dibucaine, Diclofenac (Zorvolex), Dicloxacillin, Dicycloverine, Didanosine, Diethylcarbamazine, Diethylpropion, Difenoxin, Dificid (Fidaxomicin), Diflorasone, Difloxacin, Diflucortolone Valerate, Difluprednate (Durezol), Digitek (Digoxin), Dihydroergotamine, Dihydrofolate, Dilacor (Diltiazem Hydrochloride), Dilantin (Phenytoin), Dilaudid (Hydromorphone), Diloxanide, Diltiazem, Dinoprostone (Cervidil), Diovan (Valsartan), Dipentum (Olsalazine), Diphenoxylate, Dipivefrin (Propine), Diprolene AF (Betamethasone Dipropionate), Dipropylcyclopentylxanthine, Diproqualone, Dirithromycin, Disalcid (Salsalate), Disopyramide, Ditiazem, Ditropan (Oxybutynin), Diucardin (Hydroflumethiazide), Divaplon, Docetaxel, Docusate sodium, Dodecadepsipeptides, Dolasetron (Anzemet), Dolophine (Methadone), Domperidone, Donepezil (Aricept), Dopar (Levodopa), Doribax (Doripenem), Doryx (Doxycycline Hyclate), Dostinex (Cabergoline), Doutegravir (Tivicay), Doxapram (Dopram), Doxazosin, Doxil (Doxorubicin Hcl), Doxycycline, D-Penicillamine, Dritho-Scalp (Anthralin), Dronedarone, Droperidol, Drospirenone, Drotrecogin alfa (Xigris), Droxia (Hydroxyurea), Droxidopa (Northera), Dtic-Dome (Dacarbazine), Dulaglutide, Duranest (Etidocaine HCl), Durezol (Difluprednate), Duricef (Cefadroxil), Dutasteride (Avodart), Dyloject (Diclofenac Sodium), Dynacirc (Isradipine), Dynapen (Dicloxacillin), Dyphylline, E-4031, Ebastine, Ecallantide (Kalbitor), Eculizumab (Soliris), Edarbi (Azilsartan Medoxomil), Edecrin (Ethacrynic Acid), Edex (Alprostadil), Edluar (Zolpidem Tartrate), Edoxaban (Savaysa), EDTA, Efavirenz, Effient (Prasugrel), Eflornithine, Efrotomycin, Eftifibatide (Integrilin), Efudex (Fluorouracil), EGIS-12,233, Egrifta (Tesamorelin), Eiglustat (Cerdelga), Elafin, Elaprase (Idursulfase), ELB-139, Elelyso (Taliglucerase Alfa), Elepsia (Levetiracetam), Elidel (Pimecrolimus), Eligard (Leuprolide Acetate), Eliquis (Apixaban), Elitek (Rasburicase), Ella (Ulipristal Acetate), Ellence (Epirubicin hydrochloride), Elocon (Mometasone Furoate), Eloxatin (Oxaliplatin), Elspar (Asparaginase), Eltrombopag (Promacta Eltrombopag), Eluxadoline (Viberzi), Elvitegravir (Vitekta), Emcyt (Estramustine), Emend (Aprepitant), Emgel (Erythromycin), Emtricitabine, Enablex (Darifenacin), Enalapril, Enbrel (Etanercept), Encainide, Enfuvirtide (Fuzeon), Enilospirone, Enoxacin (Penetrex), Enrofloxacin, Ensaculin, Entacapone, Entecavir (Baraclude), Entereg (Alvimopan), Entinostat, Entocort EC (Budesonide), Entyvio (Vedolizumab), Enzalutamide (Xtandi), Enzymes, Eovist (Gadoxetate Disodium), Eperezolid, Epicillin, Epicriptine, Epirubicin, Epitol, Epivir (Lamivudine), Eplerenone (Inspra), Epoetins, Epristeride, Eprobemide, Eprosartan Mesylate (Teveten), Eptapirone, Eptifibatide (Integrilin), Eraxis (Anidulafungin), Erbitux (Cetuximab), Ergomar (Ergotamine Tartrate), Ergometrine, Ergotamine, Erivedge (Vismodegib), Ertapenem, Erythromycin, Esbriet (Pirfenidone), Esketamine, ESL, Esmolol, Estramustine, Estrogens, Estrone, Estropipate, Eszopiclone, Etanercept, Etaqualone, Ethacrynic Acid (Edecrin), Ethadione, Ethamivan, Ethenzamide, Ethosuximide, Ethotoin, Ethynodiol Diacetate, Eticlopride, Etidocaine (Duranest), Etodolac (Lodine), Etomidate, Etonogestrel, Etoperidone, Etopophos (Etoposide Phosphate), Etynodiol Diacetate, Eulexin (Flutamide), Eurax (Crotamiton), Everolimus (Zortress), Evista (Raloxifene), Evoclin (Clindamycin Phosphate), Evzio, Exalgo (Hydromorphone), Exelon (Rivastigmine), Exemestane (Aromasin), Exenatide (Bydureon), Exjade, Exparel (Bupivacaine Liposome), Extina (Ketoconazole), Eylea (Aflibercept), Ezetimibe, Ezogabine (Potiga), F-15,599, Fabior (Tazarotene), Fabrazyme (Agalsidase Beta), Factive (Gemifloxacin Mesylate), Factrel (Gonadorelin), Famciclovir (Famvir), Fanapt (Iloperidone), Farydak (Panobinostat), Febuxostat (Uloric), Felbamate, Feldene (Piroxicam), Felodipine (Plendil), Fenobam, Fenofibrate (Antara), Fenoprofen Calcium (Nalfon), Fentanyl, Fesoterodine, Fetzima (Levomilnacipran), Feverall, Fibricor (Fenofibric Acid), Fidaxomicin, Filgrastim, Filipin, Finafloxacin (Xtoro), Finasteride (Propecia), Firazyr (Icatibant), Firmagon (Degarelix), Flavanoids, Flavoxate HCl (Urispas), Flecainide, Flesinoxan, Flibanserin, Flolan (Epoprostenol sodium), Flonase (Fluticasone Propionate), Florfenicol, Florinef (Fludrocortisone), Flovent (Fluticasone Propionate), Floxin (Ofloxacin), Floxuridine (Floxuridine), Fluanisone, Flubendazole, Flucloxacillin, Flucytosine, Fludrocortisone, Flumazenil (Romazicon), Flumethasone, Flunisolides, Flunitrazepam, Fluocinolone Acetonide, Fluorescein (Fluorescite), Fluorometholone, Fluoroplex (Fluorouracil), Fluoroquinolones, Fluoxymesterone (Halotestin), Fluprazine, Fluprednidene Acetate, Flurandrenolide, Flurazepam, Flurbiprofen (Ansaid), Flurithromycin, Fluspirilene, Flutamide (Eulexin), Fluticasones, FML (Fluorometholone), Focalin (Dexmethylphenidate), Folic Acid, Follistim AQ (Follitropin Beta), Folotyn (Pralatrexate Solution), Fomivirsen (Vitravene), Foradil (Formoterol Fumarate), Formoterol, Formycin A, Fortaz (Ceftazidime), Fosamprenavir Calcium (Lexiva), Fosaprepitant, Dimeglumine, Fosinopril, Fosphenytoin, Fragmin (Dalteparin), Frova (Frovatriptan Succinate), Fumarate, Fumitremorgins, Furadantin (Nitrofurantoin), Furazolidone (Furoxone), Fusidic acid, Fusilev (levoleucovorin), Fycompa (Perampanel), GABA, Gabapentin, Gabazine, GABOB, Gaboxadol, Gadavist (gadobutrol), Gadodiamide (Omniscan), Gadoteridol, Gadoversetamide, Gadoxetate Disodium, Galsulfase, Ganaxolone, Ganciclovir (Cytovene), Ganirelix, Gatifloxacin, Gazyva (Obinutuzumab), Gedocarnil, Geldanamycin, Gelnique (Oxybutynin Chloride), Gemcitabine (Gemzar), Gemifloxacin, Gemtuzumab Ozogamicin (Mylotarg), Gemzar (Gemcitabine), Gengraf (Cyclosporine), Gentamicin, Geocillin (Carbenicillin Indanyl Sodium), Geodon (Ziprasidone), Gepirone, Giazo (Balsalazide Disodium), Gilotrif (Afatinib), Glatiramer Acetate (Copaxone), Gleevec (Imatinib Mesylate), Gleostine (Lomustine), Gliclazide, Glimepiride (Amaryl), Gliotoxin, Glipizide (Glucotrol XL), Glitazones, Glucocorticoids, Glucuronic Acid, Glutarimide, Glutathione, Glutethimide, Glyburide (Micronase), Glycolipidpeptides, Glycolipids, Glycopeptides, Glycoproteins, Glycolic Acid, Glycopyrrolate (Robinul), Glycopyrronium Bromide, Glycylcyclines, Golimumab, Gonadorelin (Factrel), Gonadotropins, Gonal-F (Follitropin Alfa), Goserelin, Gramicidins A, B, and C, Granisetron (Kytril), Grepafloxacin (Raxar), Gris Peg (Griseofulvin), Guanfacine, Guanine, Guanosine, Halcinonide, Haldol (Haloperidol), Halobetasol, Halometasone, Haloperidol, Halotestin (Fluoxymesterone), Herceptin (Trastuzumab), Hetacillin, Heterocyclic Acetic Acids, Hetlioz (Tasimelteon), Hexadrol (Dexamethasone Sodium Phosphate), Histrelin Acetate (Vantas), Hivid (Zalcitabine), HMS (Medrysone), HNP-1, HNP-2, Homatropine Methylbromide, Horizant (Gabapentin Enacarbil), Humalog, Humira (Adalimumab), Hyalgan (Hyaluronate), Hyaluronate, Hyaluronic Acid, Hyaluronidases, Hycamtin (Topotecan), Hydantoins, HYDIA, Hydrazines, Hydrea (Hydroxyurea), Hydrochlor, Hydrocodone, Hydrocortisones, Hydromet, Hydromorphone, Hydroxocobalamin, Hydroxycarbamide, Hydroxydione, Hydroxyprogesterone Caproate (Makena), Hydroxysteroids, Hydroxyurea, Hyoscine, Hyoscyamine (Levsin), Hyperforin, Hytrin (Terazosin Hcl), Ibrance (Palbociclib), Ibritumomab Tiuxetan (Zevalin), Ibuprofen, Icatibant (Firazyr), Iclusig (Ponatinib), Icosapent Ethyl, Idamycin (Idarubicin), Idelalisib, Idursulfase (Elaprase), Ikarugamycin, Ilaris (Canakinumab), Ilevro (Nepafenac), Iloperidone (Fanapt), Ilotycin (Erythromycin), Iluvien (Fluocinolone Acetonide), Imatinib, Imbruvica (Ibrutinib), Imidazenil, Imidazopyridines, Imiglucerase (Cerezyme), Imipenem, Imodium (Loperamide HCl), Inapsine (Droperidol), Incivek (Telaprevir), Indacaterol (Arcapta), Indapamide, Indinavir, Indiplon, Indocin (Indomethacin), Infliximab, Ingenol Mebutate (Picato), Inlyta (Axitinib), Inspra (Eplerenone), Insulin, Intal (Cromolyn), Integrilin (Eptifibatide), Interferon alfa-2a, Interferon alfa-2b, Interferon Alfacon-1 (Infergen), Interferon beta-1a (Avonex), Interferon Beta-1b (Extavia), Interferon Gamma 1b (Actimmune), Interferons, Intuniv (Guanfacine), Invanz (Ertapenem), Invega (Paliperidone), Invirase (Saquinavir Mesylate), Iodobenzamide, Iohexol, Ionomycin, Ionsys (Fentanyl Iontophoretic), Iopamidol (Isovue-M), Iopromide (Ultravist), Ioversol (Optiray), Ioxilan (Oxilan), Ipilimumab, Ipratropium, Iprivask (Desirudin), Iproclozide, Iproniazid, Ipsapirone, Iquix (Levofloxacin), Irbesartan, Irinotecan, Isentress (Raltegravir), Isepamicin, Isocarboxazid (Marplan), Isoguvacine, Isoniazids, Isopto Carpine (Pilocarpine), Isopto Hyoscine (Scopolamine), Isotretinoin, Isradipine, Istodax (Romidepsin), Itopride, Itraconazole, Iturin A, Ivabradine (Corlanor), Ivacaftor (Kalydeco), Ivermectin, Ixabepilone (Ixempra), Izba (Travoprost), J-113,397, Jadenu (Deferasirox), JDTic, Jetrea (Ocriplasmin), Jevtana (Cabazitaxel), JNJ-7777120, Josamycin, JTC-801, Juxtapid (Lomitapide), K-252a, K-252b, Kalydeco (Ivacaftor), Kasugamycin, Kavalactones, Kazano (Alogliptin), Keflex (Cephalexin), Kendomycin, Kepivance (Palifermin), Keppra (Levetiracetam), Ketalar (Ketamine Hydrochloride), Ketamine, Ketanserin, Ketek (Telithromycin), Ketobemidone, Ketocaine, Ketoconazole, Ketolides, Ketoprofen (Orudis), Ketorolac, Ketorolac Tromethamine (Acular), Ketotifen, Keytruda (Pembrolizumab), Kinevac (Sincalide), Kinlytic (Urokinase), Kirromycin, Kitasamycin, Klaron (Sodium Sulfacetamide), Klonopin (Clonazepam), Koate (Antihemophilic Factor), Konyne (Factor IX Complex), Korlym (Mifepristone), Krystexxa (Pegloticase), Kuric (ketoconazole), Kuvan (Saproterin Dihydrochloride), Kybella, Kynamro (Mipomersen Sodium), Kyprolis (Carfilzomib), Kytril (Granisetron), Labetalol, Lac-Hydrin (Lactic Acid), Lacosamide (Vimpat), Lactoferricin B, Lafutidine, Lamivudine (3TC), Lanoxin (Digoxin), Lanreotide (Somatuline), Laronidase (Aldurazyme), Lasix (Furosemide), Lastacaft (Alcaftadine), Latamoxef, Latanoprost, Latisse (Bimatoprost), Latuda (Lurasidone HCl), Lazanda (Fentanyl), Ledipasvir, Leflunomide, Lemtrada (Alemtuzumab), Lenalidomide (Revlimid), Lenperone, Lenvatinib (Lenvima), Lepirudin (Refludan), Leptomycin A, Leptomycin B, Letairis (Ambrisentan), Leucovorin, Leukine (Sargramostim), Leuprolide, Leuprorelin, Levaquin (Levofloxacin), Levbid (Hysocyamine Sulfate), Levemir (Insulin Detemir), Levetiracetam, Levitra (Vardenafil HCl), Levobunolol (Betagan), Levocetirizine, Levofloxacin, Levomefolate Calcium, Levomethadyl Acetate (Orlaam), Levomilnacipran, Levonorgestrel, Levsin (Hyoscyamine), Lexiscan (Regadenoson), Lexiva (Fosamprenavir Calcium), Lexxel (Enalapril Maleate-Felodipine), Licarbazepine, Lidex, Lidocaine, Linaclotide (Linzess), Linagliptin (Tradjenta), Lincomycin, Lincosamides, Linezolid, Lipiarmycins, Lipids, Lipitor (Atorvastatin Calcium), Lipodepsinonapeptides, Lipofen (Fenofibrate), Lipoglycopeptides, Lipopeptides, Lipopolysaccharides, Liraglutide, Lisdexamfetamine, Lisinopril (Zestril), Lisuride, Livalo (Pitavastatin), Lodoxamide Tromethamine (Alomide), Lofentanil, Lofepramine, LoKara (Desonide), Lomefloxacin, Lomitapide (Juxtapid), Lomustine, Loperamide, Lopinavir, Lorabid (Loracarbef), Loratadine, Lorazepam, Lorediplon, Lotemax (Loteprednol Etabonate), Lotensin (Benazepril), Loteprednol Etabonate (Lotemax), Lotronex (Alosetron), Lovastatin (Advicor), Lovaza, Lozol (Indapamide), Lubiprostone (Amitiza), Lucentis (Ranibizumab), Lucinactant (Surfaxin), Lufyllin (Dyphylline), Lumacaftor, Lumigan (Bimatoprost), Lumizyme (Alglucosidase Alfa), Lunesta (Eszopiclone), Lupron (Leuprolide Acetate), Lurasidone, LY-379,268, Lymecycline, Lysostaphin, Macrobid (Nitrofurantoin), Macrolides, Macugen (Pegaptanib Sodium), Magnamycin, Magnevist (Gadopentetate Dimeglumine), Makena (Hydroxyprogesterone Caproate), Malathion (Ovide), Maleic Acid, Mandol (Cefamandole), Maraviroc (Selzentry), Marcaine (Bupivacaine and Epinephrine), Marplan (Isocarboxazid), Matulane (Procarbazine), Mavik (Trandolapril), Mavoglurant, Maxaquin (Lomefloxacin), Maxidone, MBQ, MEA, Mebaral (Mephobarbital), Mebendazole (Vermox), Mebicar, Meclocycline, Medrol (Methylprednisolone), Medroxyprogesterone Acetate (Provera), Medrysone (HMS), Mefoxin (Cefoxitin), Megace (Megestrol Acetate), Meglumine Iotroxate, Mekinist (Trametinib), Melatonin, Meloxicam (Mobic), Melperone, Menadione, Mepenzolate Bromide (Cantil), Meperidine, Mephenytoin, Mephobarbital (Mebaral), Mephyton (Phytonadione), Mepivacaine (Carbocaine), Meprobamate, Mepron (Atovaquone), Meropenem, Mestinon (Pyridostigmine), Mesuximide, Metacycline, Metadate (Methylphenidate), Metampicillin, Metaxalone (Skelaxin), Methacholine Chloride (Provocholine), Methadone (Dolophine), Methaqualone, Metharbital, Methazolamide, Methergine (Methylergonovine Maleate), Methicillin, Methocarbamol (Robaxin), Methohexital Sodium (Brevital Sodium), Methotrexate, Methoxsalen (Uvadex), Methsuximide (Celontin), Methyldopa, Methylin (Methylphenidate HCl), Methylnaltrexone Bromide (Relistor), Methylphenidate, Methylprednisolone, Methyltestosterone (Testred), Methysergide maleate (Sansert), Metipranolol (Optipranolol), Metirosine, Metoclopramide, Metolazone (Mykrox), Metopirone (Metyrapone), Metozolv ODT (Metoclopramide), Metreleptin (Myalept), Metrodin (Urofollitropin), Metvixia (Methyl Aminolevulinate), Mevacor (Lovastatin), Mevastatin, Mezlocillin, Micafungin Sodium (Mycamine), Midamor (Amiloride), Midecamycin, Midodrine, Mifeprex (Mifepristone), Migranal (Dihydroergotamine Mesylate), Milnacipran, Milrinone (Primacor IV), Minaxolone, Mineralocorticoids, Minipress (Prazosin HCl), Minocin (Minocycline), Miocamycin, Miostat (Carbachol), Mirabegron (Myrbetriq), Miradon (Anisindione), Mircera, Misoprostol, Mithracin (Plicamycin), Mitigare (Colchicine), Mitomycins, Mitoxantrone (Novantrone), Mivacron (Mivacurium Chloride), Mivazerol, MMQ, Moban (Molindone Hydrochloride), Mobic (Meloxicam), Mocetinostat, Moclobemide, Modafinil, Moexipril, Mometasone, Monobactams, Monoclate-P (Antihemophilic Factor), Monodox (Doxycycline), Moracizine, Mosapride, Moxatag (Amoxicillin), Moxeza, MPPP, Multaq (Dronedarone), Mupirocin, Mutamycin (Mitomycin), Myalept (Metreleptin), Mycamine (Micafungin Sodium), Mycobutin (Rifabutin), Mycophenolate Mofetil, Mycophenolic Acid (Myfortic), Mycosubtilin, Myfortic, Mykrox (Metolazone), Mylotarg, Myrbetriq (Mirabegron), Mysoline (Primidone), Mytelase, Nabilone, Nabumetone, Nafadotride, Nafarelin, Nafcillin, Nalidixic Acid, Naloxone, Naltrexone, Naluzotan, Naproxen, Narasin, Naropin (Ropivacaine Hcl), NAS, Nasacort AQ (Triamcinolone Acetonide), Nasalcrom (Cromolyn Sodium), Nasalide (Flunisolide), Nascobal (Cyanocobalamin), Nasonex (Mometasone Furoate), Natacyn (Natamycin), Natalizumab (Tysabri), Nateglinide (Starlix), Natrecor (Nesiritide), Natroba (Spinosad), Navelbine (Vinorelbine Tartrate), Necopidem, Nedocromil, Nefazodone, Nelfinavir Mesylate (Viracept), Nembutal (Pentobarbital), Nemonapride, Neocarzinostatin, Neoral (Cyclosporine), Neosporin, Neostigmine, Nepafenac (Ilevro), Nesacaine (Chloroprocaine), Nesina (Alogliptin), Nesiritide, Netropsin, Netupitant, Neulasta (Pegfilgrastim), Neumega (Oprelvekin), Neupogen (Filgrastim), Nevanac (Nepafenac), Nevirapine, Nexavar (Sorafenib), Nexterone (Amiodarone), Niacin, Nialamide, Niaprazine, Nicarbazin, Nicardipine, Niclosamide, Nicocodeine, Nicomorphine, Nicotinamide, Nicotinic Acid, Nifedipine, Nikethamide, Nilandron (Nilutamide), Nilotinib (Tasigna), Nimbex (Cisatracurium Besylate), Nimetazepam, Nimodipine (Nimotop), Nintedanib, Nisin, Nisoldipine (Sular), Nitazoxanide (Alinia), Nitisinone (Orfadin), Nitrazepam, Nitrofurans, Nitrofurantoins, Nitromethaqualone, Nitroxazepine, Nivolumab (Opdivo), Nizoral (Ketoconazole), Nogalamycin, Nonactin, Nonbenzodiazepines, Nordazepam, Norethindrones, Norethisterone, Norfloxacin, Norgestimate, Norgestrel, Noroxin (Norfloxacin), Norpace (Disopyramide Phosphate), Norpethidine, Nor-QD (Norethindrone), Nortilidine, Norvasc (Amlodipine Besylate), Norvir (Ritonavir), Nourseothricin sulfate, Novantrone (Mitoxantrone), Novobiocins, Noxafil (Posaconazole), Nplate (Romiplostim), NSI-189, Nubarene, Nuedexta, Nulojix (Belatacept), Numorphan (Oxymorphone), Nuromax (Doxacurium Chloride), Nuvigil (Armodafinil), Nymalize (Nimodipine), Nystatin, Obinutuzumab, Ocella, Ochratoxins, Ocinaplon, Octhilinone, Octreotide, Ocuflox (Ofloxacin), Ofatumumab (Arzerra), Ofev (Nintedanib), Ofirmev (Acetaminphen), Ofloxacin, Ogen (Estropipate), Olaparib (Lynparza), Oleandomycin, Oleptro (Trazodone Hydrochloride), Oligomers, Oligomycins, Oligopeptides, Olmesartan Medoxomil (Benicar), Olodaterol, Olopatadine, Olsalazine, Olux (Clobetasol Propionate), Olysio (Simeprevir), Omacetaxine Mepesuccinate (Synribo), Omalizumab (Xolair), Ombitasvir, Omnaris (Ciclesonide), Omnicef (Cefdinir), Omontys (Peginesatide), Oncaspar (Pegaspargase), Ondansetron, Onfi (Clobazam), Onglyza (Saxagliptin), Onsolis (Fentanyl Buccal), Ontak (Denileukin Diftitox), Opdivo (Nivolumab), Oprelvekin (Neumega), Opticrom (Cromolyn Sodium), Optipranolol (Metipranolol), Optivar (Azelastine hydrochloride), Oracea (Doxycycline), Orap (Pimozide), Orbactiv, Orencia (Abatacept), Orfadin (Nitisinone), Oritavancin, Orkambi, Orlaam (Levomethadyl Acetate), Orlistat (Xenical), Orudis (Ketoprofen), Oseltamivir, Otezla (Apremilast), Otrexup (Methotrexate), Ovide (Malathion), Oxacarbazepine, Oxacillin, Oxaliplatin, Oxandrin (Oxandrolone), Oxatomide, Oxazepam, Oxazolidinediones, Oxazolidinones, Oxcarbazepine (Trileptal), Oxilan (Ioxilan), Oxitriptan, Oxitropium Bromide, Oxolinic acid, Oxosiloxanes, Oxybuprocaine, Oxybutynin, Oxycodone, Oxyfedrine, Oxymetholone, Oxymorphone, Oxytetracycline, Oxytocics, Paclitaxel, Pagoclone, Palbociclib (Ibrance), Palifermin (Kepivance), Paliperidone, Palivizumab (Synagis), Palonosetron, Panadiplon, Panitumumab (Vectibix), Panobinostat (Farydak), Papains, Parabens, Paracetamol, Parafon Forte (Chlorzoxazone), Paramethadione, Paraplatin (Carboplatin), Paraxanthine, Paraxazone, Pardoprunox, Paritaprevir, Parlodel (Bromocriptine Mesylate), Pasireotide, Patulin, Pazinaclone, Pediocins, Pefloxacin, PEG comounds, Pegademase Bovine (Adagen), Peganone (Ethotoin), Pegaptanib, Pegfilgrastim (Neulasta), Peginesatide (Omontys), Peginterferon alfa-2a (Pegasys), Peg-Intron (Peginterferon alfa-2b), Pegvisomant (Somavert), Pegylated interferon alpha 2a, Pegylated interferon alpha 2b, Pemetrexed (Alimta), Pemirolast, Pemoline (Cylert), Penams, Penciclovir (Denavir), Penems, Penetrex (Enoxacin), Penicillin G, Penicillin V, Penicillin VK, Penicillins, Penicillin-Streptomycin, Penimepicycline, Penlac (Ciclopirox), Pentobarbital, Pentothal (Thiopental Sodium), Pentoxifylline, PEPAP, Peptide Hormones, Peptidoglycans, Peramivir (Rapivab), Perampanel, Perindopril Erbumine (Aceon), Periostat (Doxycycline), Perjeta (Pertuzumab), Perospirone, Pertuzumab (Perjeta), Pethidine, Pethidinic Acid, PGLa, Phenacemide, Phenadoxone, Phenazone, Pheneturide, Phenobarbital, Phenobarbitone, Phenoxymethylpenicillin, Phenoxymethylpenicillinic Acid, Phensuximide, Phenylacetate, Phenylacetic acid, Phenylethylmalonamide, Phenylpiperazines, Phenylpiperidines, Phenytoin, Pheromones, Phleomycins, Phosphomycin, Photofrin (Porfimer Sodium), Physostigmine Salicylate, Phytomenadione, Picato (Ingenol Mebutate), Pilocarpine (Isopto Carpine), Pimaricin, Pimavanserin, Pimecrolimus, Piminodine, Pimozide, Pinazepam, Pioglitazone, Pipadone, Pipamperone, Pipemidic acid, Piperacillin, Pirarubicin, Pirfenidone, Piritramide, Pirlimycin, Piroxicam, Pitavastatin (Livalo), Pitocin (Oxytocin), Pitressin (Vasopressin), Pivampicillin, Pivhydrazine, Platensimycin, Plavix (Clopidogrel Bisulfate), Plenaxis (Abarelix), Plendil (Felodipine), Pletal (Cilostazol), Pleuromutilins, Plicamycin (Mithracin), Podofilox, Polyene Antibiotics, Polymyxin B, Polymyxins, Polypeptides, Polysaccharides, Polysporin, Pomalidomide (Pomalyst), Ponatinib (Iclusig), Ponstel (Mefenamic Acid), Poractant Alfa (Curosurf), Porfimer Sodium (Photofrin), Posaconazole, Posizolid, Potassium Clavulanate, Potiga (Ezogabine), Povidone, Pradaxa (Dabigatran Etexilate Mesylate), Pralatrexate (Folotyn), Pralidoxime, Pramlintide Acetate (Symlin), Prasugrel, Pravachol (Pravastatin Sodium), Pravastatin, Prazepam, Praziquantel (Biltricide), Prazosin HCl (Minipress), Prednicarbate, Prednisolone, Prednisone, Pregabalin, Pregnanes, Pregnanolone, Pregnenolone, Pregnyl (Chorionic Gonadotropin), Prepidil (Dinoprostone), Prezista (Darunavir), Priftin (Rifapentine), Prilocaine, Primacor IV (Milrinone), Primidone, Prinivil (Lisinopril), Pristinamycin IIA, Pristinamycins, Proamatine (Midodrine), Procainamide, Procaine, Procaine Benzylpenicillin, Procarbazine, Procardia (Nifedipine), Procaterol, Procrit (Epoetin Alfa), Prodine, Progabide, Progesterones, Progestogens, Prograf (Tacrolimus), ProHance (Gadoteridol), Prolastin (Alpha), Prolensa (Bromfenac), Proleukin (Aldesleukin), Prolia (Denosumab), Promacta (Eltrombopag), Pronestyl (Procainamide), Propafenone, Proparacaine, Propine (Dipivefrin), Propiram, Propizepine, Proplex-T (Factor IX Complex), Propoxyphene (Darvon), Propylthiouracil, Proquin XR (Ciprofloxacin Hcl), Proscar (Finasteride), Prostacyclins, Prostaglandin E1 and E2, Prostaglandins, Prostigmin (Neostigmine), Proteins, Protopic (Tacrolimus), Protropin (Somatrem), Provera (Medroxyprogesterone Acetate), Provigil (Modafinil), Provocholine (Methacholine), Proxymetacaine, Prucalopride, Pryamide, Pulmozyme (Dornase alfa), Puromycins, Pyrazinamide, Pyrazinecarboxamide, Pyrazolopyrimidines, Pyridostigmine (Mestinon), Pyrimidinediones, Pyrovalerone, Pyrrolidines, QH-II-66, Qnasl (Beclomethasone Dipropionate), Quelicin (Succinylcholine), Quillivant XR (Methylphenidate), Quinapril, Quinaprilat, Quinolones, Quinupristin, Quixin (Levofloxacin), Qvar (Beclomethasone Dipropionate HFA), Raclopride, Radezolid, Radicicol, Raloxifene (Evista), Raltegravir, Ramelteon, Ramipril, Ramoplanin, Ramucirumab, Ranbezolid, Ranexa (Ranolazine), Ranibizumab (Lucentis), Rapacuronium (Raplon), Rapaflo, Rapamune (Sirolimus), Rapamycin, Rapastinel, Rapivab (Peramivir), Raptiva (Efalizumab), Rasburicase (Elitek), Raspberry Ketone, Rauwolscine, Raxar (Grepafloxacin), Rebeccamycin, Rebetol, Rebif (Interferon beta-1a), Refacto (Antihemophilic Factor), Refludan (Lepirudin), Regadenoson (Lexiscan), Reglan (Metoclopramide), Regorafenib (Stivarga), Regranex (Becaplermin), Relafen (Nabumetone), Relenza (Zanamivir), Relistor (Methylnaltrexone), Remacemide, Remicade (Infliximab), Remifentanil, Remoxipride, Renanolone, Renese (Polythiazide), Renova (Tretinoin), Renzapride, ReoPro (Abciximab), Repaglinide (Prandin), Reproterol, Requip (Ropinirole), Rescriptor (Delavirdine), Rescula (Unoprostone isopropyl), Reserpine, Restasis (Cyclosporine), Restoril (Temazepam), Retapamulin (Altabax), Retavase (Reteplase), Retigabine (Trobalt), Retin-A (Tretinoin), Retinoids, Retisert (Fluocinolone Acetonide), Retrovir (Zidovudine), Revatio (Sildenafil Citrate), Reveromycin A, Revia (Naltrexone), Revlimid (Lenalidomide), Revospirone, Reyataz (Atazanavir Sulfate), Rheumatrex (Methotrexate), Rhinocort Aqua (Budesonide), Ribavirin, Riboflavin, Ricobendazole, Ridaura (Auranofin), Rifabutin, Rifampicin, Rifampin, Rifamycins, Rifapentine (Priftin), Rifaximin (Xifaxan), Rilonacept (Arcalyst), Rimexolone (Vexol), Riociguat (Adempas), Risperidone, Ristomycin, Ritalin (Methylphenidate), Ritonavir (Norvir), Rituxan (Rituximab), Rivaroxaban, Rivastigmine, Rol5-4513, RO-4491533, Robaxin (Methocarbamol), Robinul (Glycopyrrolate), Rocephin (Ceftriaxone), Rocuronium, Rofecoxib (Vioxx), Roflumilast, Rokitamycin, Rolipram, Rolitetracycline, Romazicon (Flumazenil), Romidepsin (Istodax), Romiplostim (Nplate), Ropinirole, Ropivacaine (Naropin), Rosiglitazone, Rosiglitazone Maleate (Avandia), Rosuvastatin Calcium (Crestor), Roxatidine, Roxithromycin, Rozerem, Rufinamide (Banzel), RWJ-51204, Ryanodex (Dantrolene Sodium), Rythmol (Propafenone), S-14,506, S-14671, Sacubitril, Saizen (Somatropin), Salagen (Pilocarpine), Salicyclic Acid, Salicylamide, Salicylates, Salinomycin, Salsalate (Disalcid), Salvinorin A, Samidorphan, Samsca (Tolvaptan), Sanctura (Trospium), Sancuso (Granisetron), Sandimmune (Cyclosporine), Sandostatin (Octreotide Acetate), Sangivamycin, Sansert (Methysergide maleate), Saproterin, Saquinavir (SQV), Sarcosine, Sargramostim (Leukine), Saripidem, Sarmazenil, Savaysa (Edoxaban), Savella (Milnacipran), Saxagliptin, SB-205,384, Scopolamine, Secobarbital, SecreFlo (Secretin), Sectral (Acebutolol), Secukinumab (Cosentyx), Seletracetam, Selzentry (Maraviroc), Sensorcaine (Bupivacaine), Serax (Oxazepam), Sermorelin Acetate, Seroquel (Quetiapine Fumarate), Serzone (Nefazodone), SH-053-R-CH3-2′F, Signifor (Pasireotide Diaspartate), Sildenafil, Silodosin (Rapaflo), Siltuximab (Sylvant), Simeprevir, Simponi Aria (Golimumab), Simulect (Basiliximab), Simvastatin, Sincalide (Kinevac), Sinefungin, Singulair (Montelukast Sodium), Sirolimus, Sitagliptin, Sitavig (Acyclovir Buccal), Sivextro (Tedizolid Phosphate), Skelaxin (Metaxalone), Sklice (Ivermectin), SL-651,498, Sodium Sulfacetamide, Sodium Thiopental, Sofosbuvir, Solifenacin, Soliris (Eculizumab), Solithromycin, Solodyn (Minocycline), Somatostatins, Somatotropins, Somatropins, Somatuline Depot (lanreotide), Somavert (Pegvisomant), Sonata (Zaleplon), Soolantra (Ivermectin), Sorafenib (Nexavar), Sordarin, Sovaldi (Sofosbuvir), Sparfloxacin, Spectinomycin (Trobicin), Spectinomycins, Spectracef (Cefditoren Pivoxil), Spinosad, Spiperone, Spiramycins, Spiriva, Spirodecanedione, Spironolactone, Spiroxatrine, Sporanox (Itraconazole), Spriamycin, Sprix (Ketorolac Tromethamine), Sprycel (Dasatinib), Starlix (Nateglinide), Statins, Staurosporine, Stavudine, Staxyn (Vardenafil), Stelara, Stendra (Avanafil), Steroids, Stimate (Desmopressin Acetate), Stivarga (Regorafenib), Streptogramins, Streptomycin, Streptonigrin, Streptozocin (Zanosar), Stromectol (Ivermectin), Succinimides, Succinylcholine Chloride (Anectine), Succinylsulfathiazole, Sufentanil, Sular (Nisoldipine), Sulbactam, Sulfabenzamide, Sulfacetamide, Sulfinpyrazone (Anturane), Sulfonamides, Sulfonylureas, Sulochrin, Sulpiride, Sultopride, Sumanirole, Sumycin (Tetracycline), Sunitinib Malate (Sutent), Suprax (Cefixime), Suproclone, Suramin, Surfactin, Surfaxin (Lucinactant), Suriclone, Survanta (Beractant), Sustiva (Efavirenz), Sutent (Sunitinib), Sutezolid, Suvorexant, Suxamethonium, SX-3228, Sylvant (Siltuximab), Symlin (Pramlintide Acetate), Synagis (Palivizumab), Synalar (Fluocinolone Acetonide), Synarel (Nafarelin Acetate), Synribo (Omacetaxine Mepesuccinate), Synvisc (Hylan G-F 20), Syringomycin E, Tacrolimus, Tadalafil, Tafluprost (Zioptan), Talampicillin, Tambocor (Flecainide), Tamiflu (Oseltamivir), Tandospirone, Tanespimycin, Tanzeum (Albiglutide), Tao (Troleandomycin), Tasigna (Nilotinib), Tasimelteon (Hetlioz), Tasmar (Tolcapone), Taxol (Paclitaxel), Taxotere (Docetaxel), Tazarotene (Tazorac), Tazobactam, Tecfidera (Dimethyl Fumarate), Tecloftalam, Tedizolid, Tegretol (Carbamazepine), Teicoplanin, Telavancin (Vibativ), Telbivudine (Tyzeka), Telithromycin, Temafloxacin, Temazepam, Temocillin, Temovate (Clobetasol Propionate), Temozolomide (Temodar), Temsirolimus (Torisel), Tenecteplase (Tnkase), Tenex (Guanfacine), Teniposide (Vumon), Tenofovir, Tenuate (Diethylpropion), Terazosin, Teriflunomide, Teriparatide, Tesamorelin (Egrifta), Teslac (Testolactone), Tessalon (Benzonatate), Testosterone, Tetrabenazine (Xenazine), Tetracaine, Tetracyclines, Thalitone (Chlorthalidone), Thalomid (Thalidomide), Theanine, Theobromine, Theophylline, Thiamphenicol, Thiazolidinediones, Thiolutin, Thiopental, Thiostrepton, Thrombate (Antithrombin), Thrombin, Thromboxanes, Thujone, Thyrogen (Thyrotropin Alfa), Thyroid Hormones, Tiagabine, Tiamulin, Tianeptine, Tiapride, Tiazac (Diltiazem Hel), Tiazesim, Ticarcillin, Tigan (Trimethobenzamide), Tigecyclines, Tilade (Nedocromil), Tiospirone, Tiotropium Bromide (Spiriva), Tioxolone, Tipranavir, Tirilazad, Tivicay (Doutegravir), Tivorbex (Indomethacin), Tocainide, Tocilizumab (Actemra), Tofacitinib Tablets (Xeljanz), Tolazamide (Tolinase), Tolbutamide, Tolcapone (Tasmar), Tolectin (Tolmetin Sodium), Tolmetin, Toloxatone, Tolvaptan, Tonocard (Tocainide), Toposar, Topotecan, Toradol (Ketorolac Tromethamine), Torezolid, Torisel (Temsirolimus), Torsemide, Tositumomab (Bexxar), Toviaz (Fesoterodine Fumarate), Tracrium (Atracurium Besylate), Tradjenta (Linagliptin), Trametinib (Mekinist), Trandate (Labetalol), Trandolapril (Mavik), Tranxene (Clorazepate Dipotassium), Trastuzumab (Herceptin), Trasylol (Aprotinin), Travatan (Travoprost), Trazodone, Trelstar, Trental (Pentoxifylline), Trexall (Methotrexate), Triamcinolone Acetonide, Triazoles, Tricor (Fenofibrate), Tridione (Trimethadione), Triesence, Trifluperidol, Trifluridine (Viroptic), Triglide (Fenofibrate), Trileptal (Oxcarbazepine), Trimethobenzamide, Trimethoprims, Trimethylglycine, Trimetozine, Triptorelin, Tritoqualine, Trobicin (Spectinomycin), Troleandomycin, Tropicamide, Trospium, Trovafloxacin, Tudorza Pressair (Aclidinium Bromide), Tunicamycin C2, Tunicamycins, Tybost (Cobicistat), Tygacil (Tigecycline), Tylosin, Tysabri (Natalizumab), Tyzeka (Telbivudine), UDCA, Ulipristal Acetate, Ultiva (Remifentanil), Ultravist (Iopromide), Umespirone, Univasc (Moexipril), Unoprostone isopropyl (Rescula), Urapidil, Ureas, Urecholine, Ureidopenicillins, Urispas (Flavoxate), Urofollitropin (Fertinex), Uroxatral (Alfuzosin), URSO, USAN, Ustekinumab, Uvadex (Methoxsalen), Valaciclovir (Valtrex), Valcyte (Valganciclovir Hel), Valerenic Acid, Validolum, Valinomycin, Valnemulin, Valnoctamide, Valproates, Valpromide, Valrubicin (Valstar), Valsartan (Diovan), Valtrex (Valacyclovir), Valtropin (Somatropin), Vancomycin, Vanos (Fluocinonide), Vantas (Histrelin Acetate), Vantin (Cefpodoxmine Proxetil), Vaprisol (Conivaptan), Vardenafil, Vasopressin (Pitressin), Vasotec (Enalapril), Vasovist (Gadofosveset Trisodium), Vectibix (Panitumumab), Vecuronium, Vedolizumab, Velcade (Bortezomib), Vemurafenib (Zelboraf), VePesid (Etoposide), Vermox (Mebendazole), Verteporfin (Visudyne), VESIcare (Solifenacin), Vesnarinone, Vestipitant, Vexol (Rimexolone), Viadur (Leuprolide Acetate), Vibativ (Telavancin), Viberzi (eluxadoline), Victrelis (Boceprevir), Vidaza (Azacitidine), Videx (Didanosine), Vigabatrin, Vigamox (Moxifloxacin), Viibryd (Vilazodone), Vilazodone, Vimpat (Lacosamide), Vinblastine, Vincasar PFS (Vincristine), Vinorelbine, Vioxx (Rofecoxib), Viracept (Nelfinavir Mesylate), Virazole (Ribavirin), Viread (Tenofovir Disoproxil), Virginiamycin, Virginiamycin M1, Virginiamycin S1, Viroptic (Trifluridine), Vismodegib (Erivedge), Vistide (Cidofovir), Visudyne (Verteporfin), Vitamins, Vitekta (Elvitegravir), Vitrasert (Ganciclovir), Vitravene (Fomivirsen), Vorapaxar, Vorinostat (Zolinza), VUF-6002, Vumon (Teniposide), Vynase, Vyvanse (Lisdexamfetamine), Warfarin, WAY-100,135, WAY-100,635, Wellbutrin (Bupropion), Xalatan (Latanoprost), Xamoterol, Xanthines, Xanthosines, Xarelto (Rivaroxaban), Xeljanz (Tofacitinib), Xeloda (Capecitabine), Xenazine (Tetrabenazine), Xenical (Orlistat), Xgeva (Denosumab), Xibrom (Bromfenac), Xifaxan (Rifaximin), Xigris (Drotrecogin alfa), Ximino (Minocycline), Xolair (Omalizumab), Xtandi (Enzalutamide), Xtoro (Finafloxacin), Xylocaine (Lidocaine), Xyntha (Antihemophilic Factor), Y-23684, Yohimbine, Zacopride, Zaditor (Ketotifen), Zafirlukast, Zagam (Sparfloxacin), Zalcitabine (Hivid), Zaleplon, Zalospirone, Zaltrap, Zanamivir (Relenza), Zanosar (Streptozocin), Zarontin (Ethosuximide), Zelboraf (Vemurafenib), Zemuron (Rocuronium Bromide), Zenapax (Daclizumab), Zerit (Stavudine), Zestril (Lisinopril), Zetia (Ezetimibe), Zetonna (Ciclesonide), Ziconotide (Prialt), Zidovudine, Zileuton, Zilpaterol, Zinacef (Cefuroxime), Zinecard (Dexrazoxane), Zioptan (tafluprost), Ziprasidone, Zirgan (Ganciclovir), Zithromax (Azithromycin), ZK-93423, Zocor (Simvastatin), Zofran (Ondansetron), Zoladex (Goserelin Acetate), Zolinza (Vorinostat), Zolmitriptan (Zomig), Zolpidem, Zontivity (Vorapaxar), Zoplicone, Zortress (Everolimus), Zovirax (Acyclovir), Zuplenz (Ondansetron), Zydelig (Idelalisib), Zyflo (Zileutin), Zyloprim (Allopurinol), Zymar (Gatifloxacin), Zyrtec (Cetirizine), Zyvox (Linezolid), β-Adrenergic Receptor Agonists, β-Carbolines, β-Lactamases, and β-Lactams.

Scheme 4 shows an example where x is testosterone.

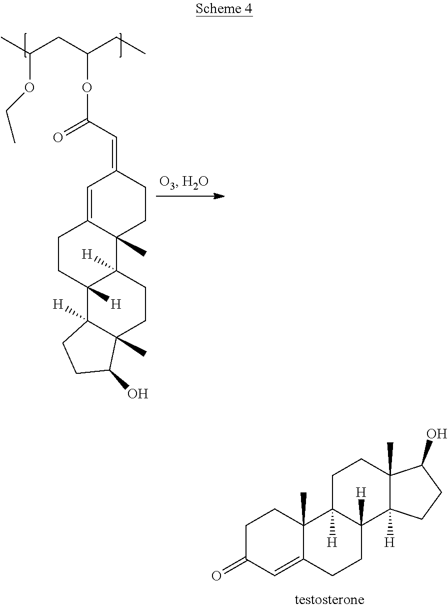

The present invention is also directed to a method of activating any of the above inactive compounds. The method comprises exposing the inactive compound with ozone for a time sufficient to activate the compound.

The various pharmaceutical prodrugs provided herein have an advantage over other prodrugs in that the speed of the release of the active compound from the R¹ group can be controlled by ozone therapy, as described, e.g., in Clavo et al., 2004, eCAM 1:93-98. For faster release of the active compound from the prodrug, more frequent and/or higher dosage of ozone therapy is indicated; for slower release, less frequent and/or lower dosage of ozone can be administered. Additionally, if R¹ comprises a specific binding agent (discussed above) that is targeted to diseased tissue, e.g., a cancer antigen binding agent such as an antibody binding site, the compound will be present in greater concentration at the diseased tissue than elsewhere, and activation of the active compound by subsequent ozone therapy will cause a comparatively low amount of activation outside the diseased tissue, with fewer side effects.

Thus, in additional embodiments, a method of treating a patient with a disease or disorder is provided. The method comprises administering any of the above compounds having an active compound that is active against the disease or disorder. In some embodiments, the disease or disorder is a cancer. In additional embodiments, intravenous ozone therapy is also administered to the patient to activate the compound. In further embodiments, R¹ is a specific binding agent that specifically binds to the disease or disorder, e.g., cancer.

It is understood that the time required to activate the inactive compound is related to the ozone concentration, where a higher ozone concentration that the inactive compound is exposed to, the greater rate of activation. Thus, where the ozone concentration is low, for example in a can of paint, the rate of activation of inactive compounds added to the paint is low, but when the paint is applied in a thin layer on a wall, the rate of activation is higher, such that the active compound, e.g., a germicide, is activated on the wall, with germicidal effect. Thus, ozone activation is an ideal slow release mechanism for an active compound that is stored in an ozone-free or ozone-depleted environment, e.g., a can of paint, spray bottle, medicine container, closed fertilizer bag, etc.

In some embodiments of these methods, the active compound is a biocide. In various embodiments, the active compound is a disinfectant.

Shown below is BAC 14, or benzyldimethyltetradecylammonium chloride, a common disinfectant. In order to make a monomer, oligomer or polymer of a disinfectant that can be activated by ozone, a derivative of BAC 14 can be made, such as compound XXXVI below.

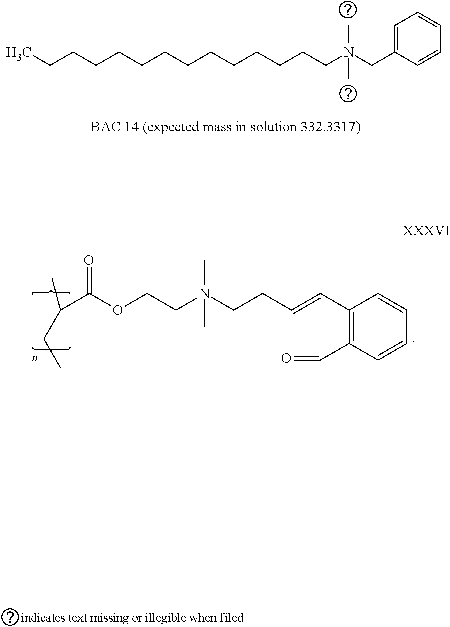

The compounds described herein can be applied to food technology. In that regard, any coating, antioxidant, preservative, flavor component, antibacterial, antifungal, or any other compound used in food preparation can be incorporated into monomers, oligomers or polymers of the present invention to provide a slow release compound that maintains its useful characteristic on or in the food or food packaging for a longer time. Such compounds are useful for meat, breads, fruit, vegetables, cheeses, or any other food, and are particularly useful for foods that can undergo oxidative reactions, and where ozone is present. Additionally, compounds that produce indicators when oxidative reactions occur, or when harmful organisms or toxins such as Salmonella spp., botulism, etc. are present e.g., a fragrance, smell, color change, fluorescent change, etc.

Examples of useful polymers that can be utilized in foods are compounds XXXVII and XXXVIII below. Compound XXXVII is a non-toxic antioxidant that yields sugar and a cellulose derivative upon ozoneolysis. Compound XXXVIII is a more nonpolar antixoidant

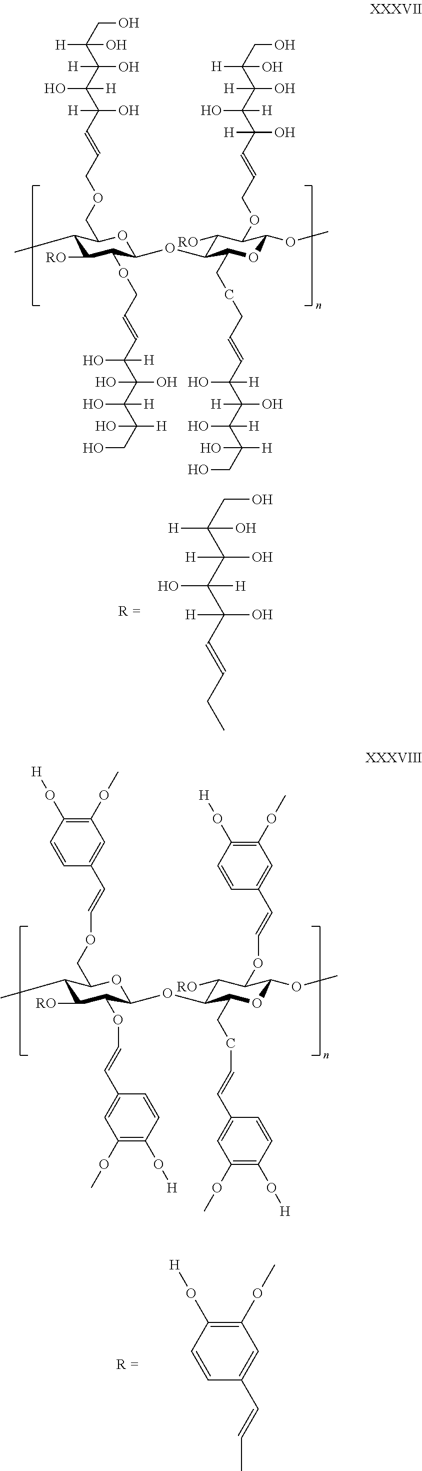

The inactive compounds provided herein are also useful for determining internal ozonolysis in a subject, for example to detect or quantify inflammation or cardiovascular disease. Thus, the present invention also provides a method of determining internal ozonolysis in a subject. The method comprises administering the above-described inactive compound to the subject, waiting for a time sufficient for the internal ozonolysis to take place, then assaying for the active compound X═O. In some embodiments, the active compound is quantified, for example in the breath, the blood, or in biopsied tissues, in order to quantify the extent of ozonolysis. Such a quantification would correlate with the extent of an implicated disease or condition, e.g., inflammation or cardiovascular disease. In various embodiments, the compound is administered into the blood stream of the subject. In other embodiments, the compound is administered to a tissue. In additional embodiments, the subject is suspected of having, or known to have, inflammation or cardiovascular disease. Examples include those of Scheme 5 and 6.

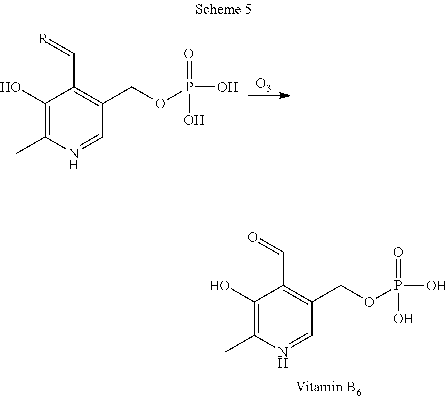

In Scheme 5, the inactive compound becomes vitamin B6, which can be easily detected in the bloodstream.

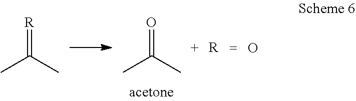

In Scheme 6, the inactive compound becomes acetone, which can be easily detected in blood or in the breath. Thus, inflammation, cardiovascular disease, or any other disease, disorder or condition where ozonolysis is implicated can be easily identified or quantitated using a breath or blood test.

In further embodiments, the active compound is formulated for environmental use. In some of these embodiments, the inactive compound is formulated in a paint or a spray, or integrated into a solid material (e.g., wallboard), or coated on the surface of a solid material.

The present invention also provides small molecules that are useful for degrading ozone, e.g., in the atmosphere, or in industrial settings where ozone is generated.

Thus in various embodiments, provided is a molecule less than 9000 mw, having a double bond that is reactive with ozone, and forms a nontoxic compound after reacting with ozone.

As used herein, “nontoxic” is a compound that is generally regarded as safe when contacted with skin, inhaled, or ingested.

These ozone degrading molecules can be any size, e.g., less than 5000 mw, less than 2000 mw, less than 1000 mw, less than 750 mw, less than 500 mw, less than 400 mw, or less than 300 mw.

In various embodiments, these molecules are not oligomeric or polymeric.

These molecules can have one or more than one moiety that reacts with ozone.

The molecules of these embodiments can have any physical properties appropriate for their application. For example, the molecule can have high water solubility or low water solubility, or high or low volatility.

Non-limiting examples of these molecules include

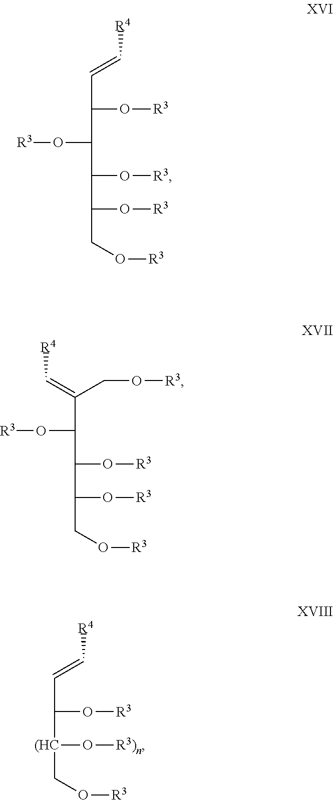

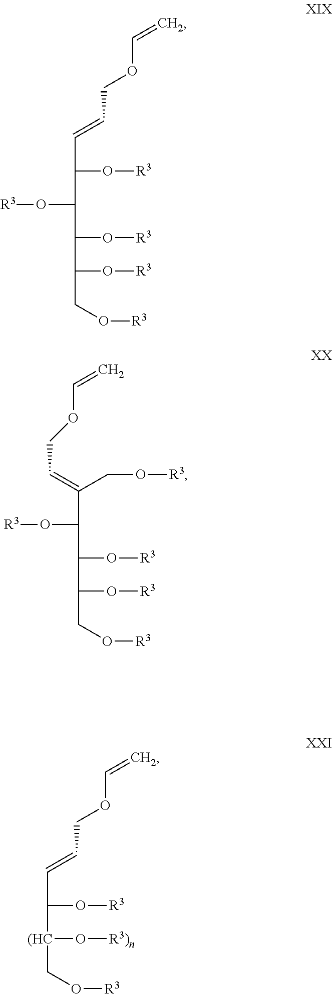

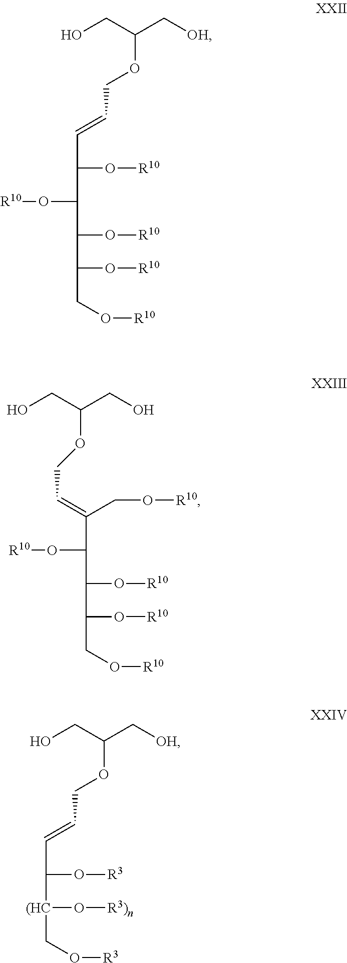

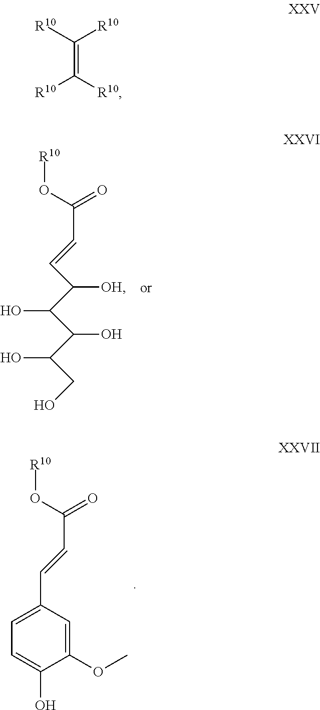

or a salt or solvate thereof, wherein:

n is an integer from 0-6,

each R³ and R⁴ is independently hydrogen, substituted or unsubstituted alkyl, substituted or unsubstituted perfluoroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, substituted or unsubstituted heteroarylalkyl, substituted or unsubstituted —(CH₂)ⱼCN, substituted or unsubstituted —(CH₂)ⱼOR⁵, substituted or unsubstituted —(CH₂)ⱼC(O)R⁵, substituted or unsubstituted —(CH₂)ⱼOC(O)R⁶, substituted or unsubstituted —(CH₂)₃C(O)OR⁵, substituted or unsubstituted —(CH₂)ⱼOC(O)OR⁵, substituted or unsubstituted —(CH₂)ⱼNR⁷R⁸, substituted or unsubstituted —(CH₂)₃C(O)NR⁷R⁸, substituted or unsubstituted —(CH₂)₃OC(O)NR⁷R⁸, substituted or unsubstituted —(CH₂)₃NR⁷C(O)R⁶, substituted or unsubstituted —(CH₂)₃NR⁷C(O)OR⁵, substituted or unsubstituted —(CH₂)ⱼNR⁷C(O)NR⁷R⁸, substituted or unsubstituted —(CH₂)ⱼS(O)ₘR⁹, substituted or unsubstituted —(CH₂)ⱼNR⁶S(O)ₘR⁹, or substituted or unsubstituted —(CH₂)ⱼS(O)ₘNR⁷R⁸,

wherein each j is independently an integer from 0 to 6; each m is independently an integer from 0 to 2; each n is independently an integer from 0 to 4;

each R⁴ may further independently be an acrylic monomer or polymer, an alkyl monomer or polymer, an epoxy monomer or polymer, a vinyl monomer or polymer or a cellulose monomer or polymer;

each R⁵ is independently hydrogen, or substituted or unsubstituted alkyl;

each R⁶ and R⁹ are independently hydrogen, or substituted or unsubstituted alkyl;

each R⁷ and R⁸ are independently hydrogen, substituted or unsubstituted alkyl, or R⁷ and R⁸, together with the N atom to which they are attached, form a 5- or 6-membered heterocyclic ring or a 5-membered heteroaryl ring; and

each R¹⁰ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl,

wherein each R³, R⁴, R⁵, R⁶, R⁷, R⁸, and R⁹ group is optionally independently substituted with 1-3 substituents, each independently alkyl, alkenyl, alkynyl, alkoxy, cycloalkyl, perfluoroalkyl, amide, amino, alkylamino, carboxylate, cyano, dialkylamino, halogen, hydroxyl, imino, nitro, oxo, sulfide, or thiol.

In various embodiments, the nontoxic compound is non-volatile, e.g., a sugar. In other embodiments, the nontoxic compound is a sugar. In other embodiments, the compound is volatile and leaves a scent. In some of these embodiments, the nontoxic compound is vanillin.

An example is provided in Scheme 5. Ethyl bromoacetate (1.0 g) is reacted with TPP (1.884 g), saturated NaHCO₃ and vanillin (1.002 g) in water at 20° C. for 1 hour with stirring, to form the intermediate compound A. That compound is reacted with base (4 g), then acid to neutralize the base, to form intermediate compound B. Upon reaction with atmospheric ozone and water for 1 hour, glyoxylic acid and vanillin, both nontoxic compounds, are formed.

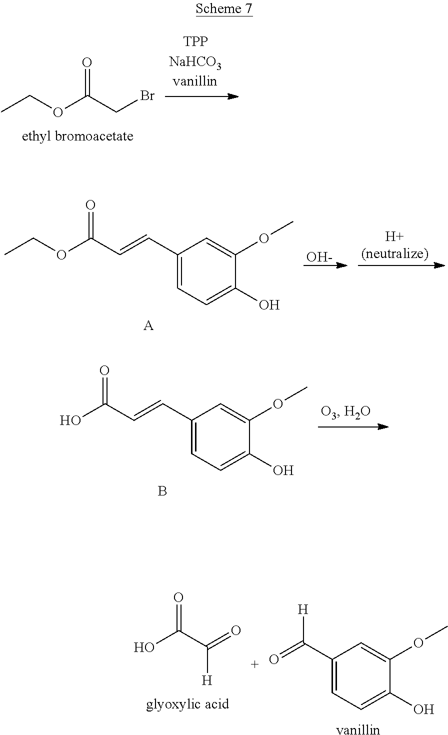

In the above Scheme 7, benzaldehyde can be substituted for vanillin, to produce glyoxylic acid and benzaldehyde.

In some embodiments, the molecule is formulated in a paint or a spray, or integrated into a solid material, or coated on the surface of a solid material.

Also provided is a method of degrading ozone. The method comprises exposing any of the above molecules to ozone for a time sufficient to degrade the ozone.

The present invention also provides additional polymeric compounds for degrading ozone, where the polymer is a sugar polymer, e.g., cellulose. Scheme 8 shows a scheme for producing an example of such a compound (Compound XXVIII). Cellulose is reacted with chloroacetic acid or choroacetic acid and the product is reacted with TPP, NaHCO₃ and glucose in the presence of water and THF to form Compound XXVIII.

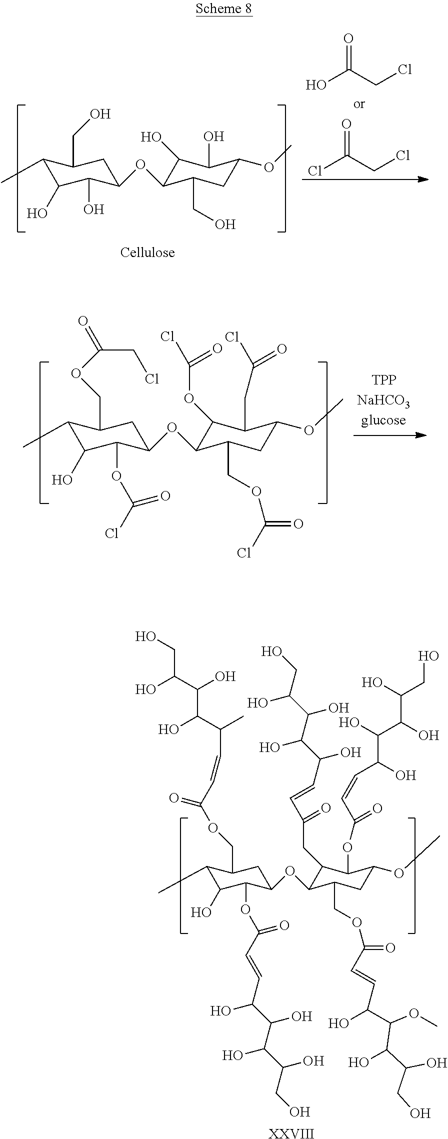

Also provided herewith are plastics that can be degraded by ozone treatment. Such plastics can be made to be biodegradable, or the recycled plastic can be treated with ozone and the oxidized product can be reused in recycled materials. These degradable plastics can be in the form of any plastic materials for which it is useful to recycle or have biodegraded (i.e., are not meant to be permanent), for example, plastic bags, milk cartons, packaging for food or other products, etc.

Examples of such ozone-degradable plastics are provided as compounds XXXIX and XXXX below.

Here are a couple polymers for the degradation of plastics through ozonolysis that can produce specific byproducts that can then be reused. The polymer on the top will produce glutaraldehyde and ortho-phthalaldehyde upon ozonolysis reactions, while the one on the bottom will only produce ortho-phthalaldehyde.

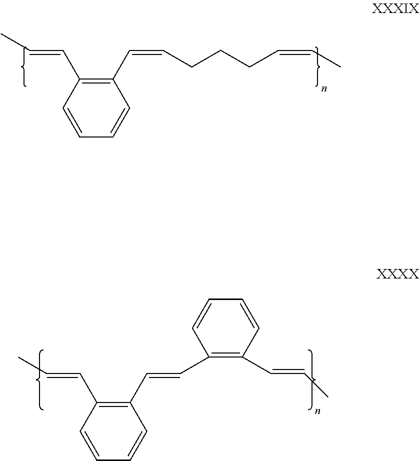

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. An inactive compound that is activated by reaction with ozone into an active compound having a carbonyl oxygen, wherein
the carbonyl oxygen in the active compound is part of an aldehyde, a ketone, a carboxylic acid, an ester, an amide, an enone, an acyl halide, an imide, an acid anhydride, a 1,3-dicarbonyl, a carbamate, a carbazide, a carbazone, a carboxylate, a cyclic imide, a formate, a furazone, a hydrazine, a hydroxamate, an isocyanate, a lactam, a lactone, a semicarbazone, a urea, a thiocarbamate, or a dithiocarbamate, and
the compound has the structure of compound I 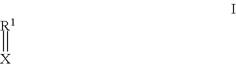
where, upon reaction with ozone, —R¹ is substituted with oxygen to form the carbonyl oxygen, forming the active compound X═O,
wherein R¹ is a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl.

2-5. (canceled)

6. The compound of claim 1, wherein X is a planar compound comprising at least three aromatic rings.

7-8. (canceled)

9. The compound of claim 6, having the structure of compound XXX, compound XXXI, compound XXXII, compound XXXIV, or compound XXXV, 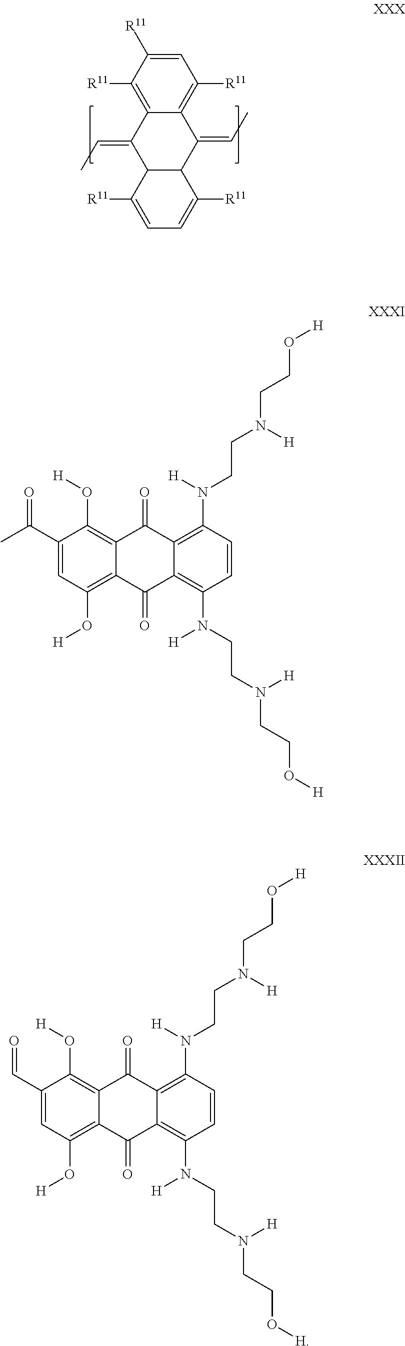 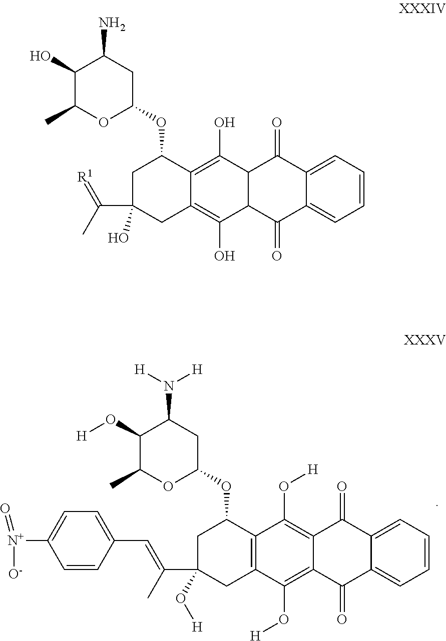
wherein R¹¹ is a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl and m is an integer from 2 to 100,000,000.

10-16. (canceled)

17. The compound of claim 1, wherein R¹ is NR² or CR², where R² is H, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl.

18. The compound of claim 1, having the structure 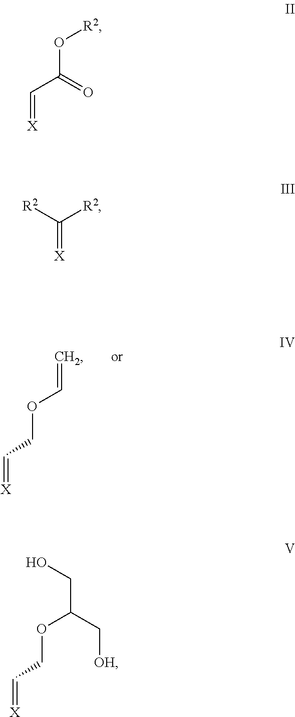
wherein each R² is independently hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl.

19. The compound of claim 1, having the structure of compound II 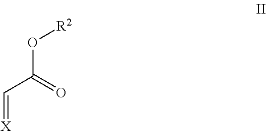
where R² is a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl.

20. The compound of claim 1, wherein R¹ comprises an oligomeric or polymeric repeat comprising more than one X.

21. The compound of claim 20, having the structure 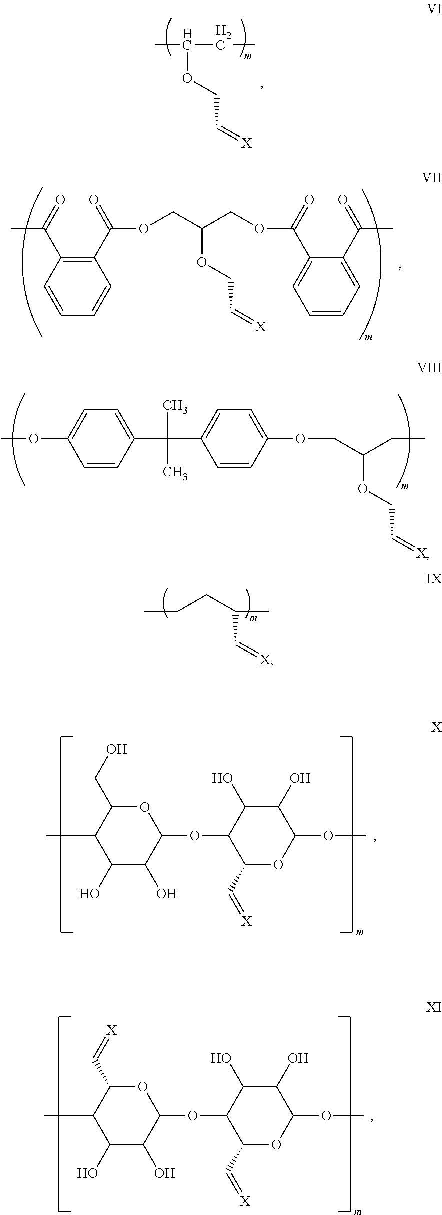 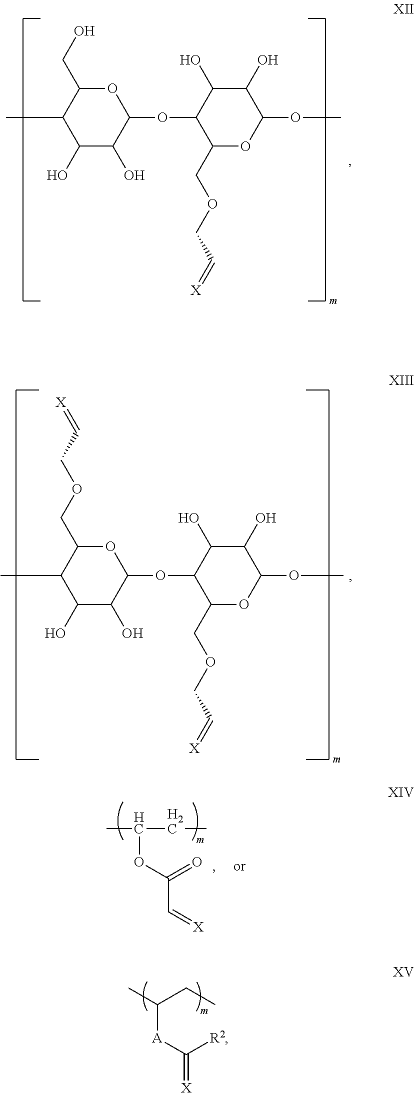
wherein
m is an integer from 2 to 100,000,000,
A is absent or a linking group selected from the group consisting of a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl, and
R² is independently hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl.

22-30. (canceled)

31. The compound of claim 1, wherein the active compound is a pharmaceutical.

32. The compound of claim 31, wherein the pharmaceutical is useful for treatment of a-cancer.

33-34. (canceled)

35. A method of treating a patient with cancer, the method comprising administering the compound of claim 32 to the patient in an amount sufficient to treat the patient.

36. The method of claim 35, further comprising administering ozone to the patient.

37-38. (canceled)

39. A method of activating the inactive compound of claim 1, the method comprising exposing the inactive compound with ozone for a time sufficient to activate the compound.

40. The method of claim 39, wherein the active compound is a biocide.

41. The method of claim 39, wherein the active compound is a pharmaceutical.

42-48. (canceled)

49. A method of treating a disease or condition in a subject, the method comprising administering the pharmaceutical compound of claim 31 to the subject at a site that is not exposed to atmospheric ozone.

50. The method of claim 49, wherein a myeloperoxidase and/or a neutrophil is present at the site.

51-55. (canceled)

56. A method of determining internal ozonolysis in a subject, the method comprising administering the compound of claim 15 to the subject, waiting for a time sufficient for the internal ozonolysis to take place, then assaying for the active compound X═O.

57-60. (canceled)

61. A molecule, having a double bond that is reactive with ozone, and forms a nontoxic compound after reacting with ozone, wherein the molecule is 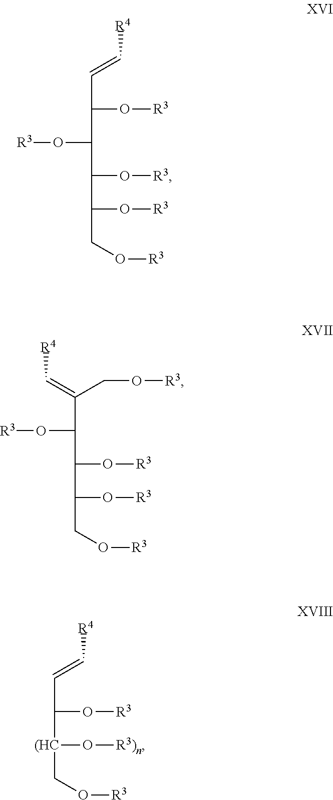 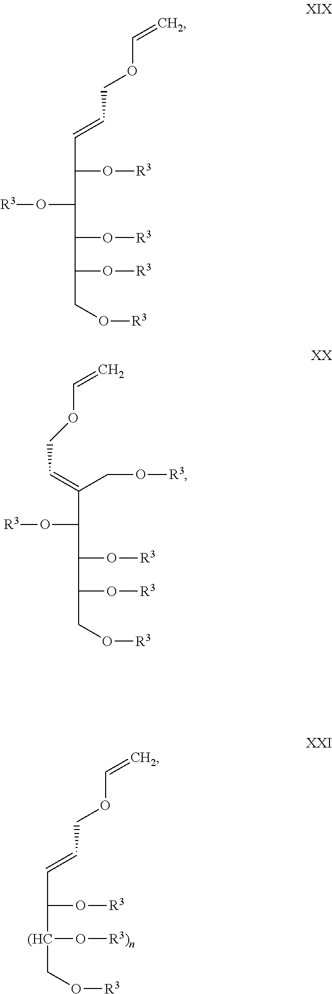 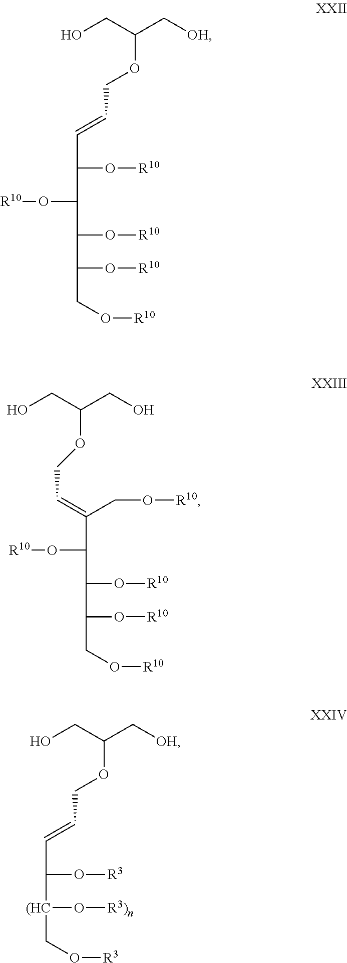 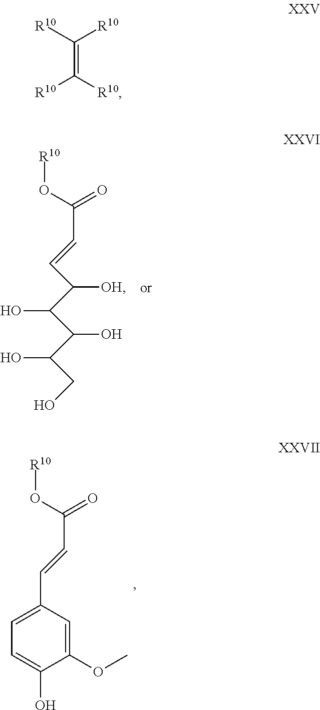
or a salt or solvate thereof, wherein:
n is an integer from 0-6,
each R³ and R⁴ is independently hydrogen, substituted or unsubstituted alkyl, substituted or unsubstituted perfluoroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, substituted or unsubstituted heteroarylalkyl, substituted or unsubstituted —(CH₂)ⱼCN, substituted or unsubstituted —(CH₂)ⱼOR⁵, substituted or unsubstituted —(CH₂)ⱼC(O)R⁵, substituted or unsubstituted —(CH₂)ⱼOC(O)R⁶, substituted or unsubstituted —(CH₂)ⱼC(O)OR⁵, substituted or unsubstituted —(CH₂)ⱼOC(O)OR⁵, substituted or unsubstituted —(CH₂)ⱼNR⁷R⁸, substituted or unsubstituted —(CH₂)ⱼC(O)NR⁷R⁸, substituted or unsubstituted —(CH₂)ⱼOC(O)NR⁷R⁸, substituted or unsubstituted —(CH₂)ⱼNR⁷C(O)R⁶, substituted or unsubstituted —(CH₂)ⱼNR⁷C(O)OR⁵, substituted or unsubstituted —(CH₂)ⱼNR⁷C(O)NR⁷R⁸, substituted or unsubstituted —(CH₂)ⱼS(O)ₘR⁹, substituted or unsubstituted —(CH₂)ⱼNR⁶S(O)ₘR⁹, or substituted or unsubstituted —(CH₂)ⱼS(O)ₘNR⁷R⁸,
wherein each j is independently an integer from 0 to 6; each m is independently an integer from 0 to 2; each n is independently an integer from 0 to 4;
each R⁴ may further independently be an acrylic monomer or polymer, an alkyl monomer or polymer, an epoxy monomer or polymer, a vinyl monomer or polymer or a cellulose monomer or polymer;
each R⁵ is independently hydrogen, or substituted or unsubstituted alkyl;
each R⁶ and R⁹ are independently hydrogen, or substituted or unsubstituted alkyl;
each R⁷ and R⁸ are independently hydrogen, substituted or unsubstituted alkyl, or R⁷ and R⁸, together with the N atom to which they are attached, form a 5- or 6-membered heterocyclic ring or a 5-membered heteroaryl ring; and
each R¹⁰ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, or substituted or unsubstituted heteroarylalkyl,
wherein each R³, R⁴, R⁵, R⁶, R⁷, R⁸, and R⁹ group is optionally independently substituted with 1-3 substituents, each independently alkyl, alkenyl, alkynyl, alkoxy, cycloalkyl, perfluoroalkyl, amide, amino, alkylamino, carboxylate, cyano, dialkylamino, halogen, hydroxyl, imino, nitro, oxo, sulfide, or thiol.

62-73. (canceled)

74. A method of degrading ozone, the method comprising exposing the molecule of claim 61 to ozone for a time sufficient to degrade the ozone.
